SEQUENCE LISTING (6 sequences)

SEQ ID NO: 1 - DNA, 25 bp, Artificial Sequence
  primer
ggagyatgtggtttaattcgaagca

SEQ ID NO: 2 - DNA, 20 bp, Artificial Sequence
  primer
agctgacgacaaccatgcac

SEQ ID NO: 3 - DNA, 22 bp, Artificial Sequence
  probe
gtttaattcgatgatacgcgag

SEQ ID NO: 4 - DNA, 19 bp, Artificial Sequence
  primer
ttaasccgacacctcacgg

SEQ ID NO: 5 - DNA, 20 bp, Artificial Sequence
  primer
agagtttgatcctggctcag

SEQ ID NO: 6 - DNA, 17 bp, Artificial Sequence
  primer
aaggaggtgwtccarcc

(19) United States
(12) Patent Application Publication
(10) Pub. No.: US 2023/0243000 A1
(43) Pub. Date: Aug. 3, 2023

(54) FMT PERFORMANCE PREDICTION TEST TO GUIDE AND OPTIMIZE THERAPEUTIC MANAGEMENT OF GVHD PATIENTS

(71) Applicant: MaaT PHARMA, Lyon (FR)

(72) Inventors: Hervé AFFAGARD, Lyon (FR); Emilie PLANTAMURA, Lyon (FR); Emmanuel PRESTAT, Lyon (FR); Cyrielle GASC, Lyon (FR); Benoit LEVAST, Lyon (FR)

(21) Appl. No.: 17/996,267

(22) PCT Filed: Apr. 16, 2021
(86) PCT No.: PCT/EP2021/059993

§ 371 Date: Oct. 14, 2022

(30) Foreign Application Priority Data
Apr. 17, 2020 (EP) 20170256.0

(51) Int. Cl. C12Q 1/689; G01N 33/68

(52) U.S. Cl. CPC C12Q 1/689; G01N 33/6863; C12Q 1/6851

(57) ABSTRACT

The present disclosure relates to a method for assessing whether a GVHD subject in need of a complementation with live microorganisms can benefit from said complementation, by analysing the subject’s microbiota and/or host parameters. Treatments for improving the status of patients identified as poor microbiotherapy responders are also provided, as well as materials and kits for performing the method according to the invention.

### FIELD OF THE INVENTION

The present invention pertains to the field of GVHD management. In particular, the present invention concerns the role of intestinal microbiota in GVHD and provides a method for determining if a patient is likely to benefit from a treatment aiming at modulating this microbiota, such as a fecal microbiota transplant (FMT), or if another treatment is necessary prior to the FMT to increase the patient’s chances of success fully respond to the FMT.

### BACKGROUND AND PRIOR ART

### GVHD

Graft versus host disease (GVHD) is a major complication of allogeneic hematopoietic stem cell transplantation (allo-HSCT) and consists of an immunologically mediated inflammatory reaction of donor immune T-cells against proteins, specifically human leukocyte antigens (HLAs), on host cells. Allo-HSCT is required for a curative intent in multiple hematologic disorders, varying from malignant diseases to genetic anaemias.

The rejection occurs when mismatch of minor histocompatibility antigens, or other reasons, trigger the donor’s immune system to attack the recipient, causing unique inflammatory disease. GVHD does not occur after autologous HSCT (cells derived from the same patient).

GVHD have two main forms depending on the symptoms’ timing:
acute GVHD (aGVHD): a clinico-pathological syndrome that occurs within 100 days post HSCT (median onset is typically 21 to 25 days after transplantation); involving mostly three organs: the skin (>80% of patients with GVHD), gastrointestinal (GI) tract (50-55%) and liver (50%). Any one organ or combination of these organs may be affected.
chronic GVHD (cGVHD) manifests with fibrotic skin disease, bronchiolitis, salivary and lacrimal gland disease, and eosinophilic fasciitis, and typically occurs more than 100 days post HSCT, often following acute GVHD.

### Current Treatment of GVHD

Management of GVHD is challenging. Immuno-suppression with corticosteroids forms the basis of first-line therapy in both acute and chronic GVHD, producing sustained responses in less than 50% of patients with aGVHD and 40-50% of patients with cGVHD, depending on initial disease severity.

For patients who do not respond well to steroids (steroid-refractory SR) or in whom steroids cannot be tapered (steroid-dependent SD), the prognosis is very poor.

A recent study described biomarkers to predict long-term outcome in steroid-resistant GVHD, including the levels of the suppressor of tumorigenicity-2 (ST2) and the regenerating-islet-derived protein 3-α (REG3α) after 1 week of systemic treatment (Major-Monfried et al., 2018).

### Importance of the Patient’s Microbiota

Patients undergoing allo-HSCT can be exposed to cytotoxic chemotherapy, total-body irradiation, immunosuppressors, and broad-spectrum antibiotics. These treatments cause dramatic alterations of the intestinal microbiota and varying degrees of damage to the intestinal mucosa, leading to breaches in host defenses.

Over the course of allo-HSCT, patients show profound shifts in microbial communities marked by a reduction of overall microbial diversity and richness, a disruption of beneficial bacteria that support host defenses (e.g., Firmicutes), and a rise in dominance of bacterial species usually subdominant, including some pathogens and pathobionts (e.g., Clostridium difficile, some Enterobacteriaceae) and multidrug-resistant (MDR) bacteria (Malard et al., 2018), associated with subsequent bacteriemia and infectious complications (Taur et al., 2012).

Current standard of care in oncology does not take into account the microbiota management, including its baseline status. However, several recent studies report that the gut microbiota is implicated in chemotherapy efficacy and toxicity through numerous mechanisms, including xenometabolism, immune interactions, and altered community structure. Without a functional microbiome, these treatments could be suboptimal (lida et al., 2013; Alexander et al., 2017; Ma et al., 2019).

In allo-HSCT patients, the diversity of the gut microbiota plays a key role in overall survival after allo-HSCT (Malard et al., 2018), and in GVHD patient outcome (Taur et al, 2014, Peled et al., 2020). Indeed, loss of microbiota diversity was observed to be associated with more pronounced gastrointestinal GVHD (Holler et al., 2014). Several studies reported that disrupted microbiota (e.g. loss of diversity, domination by single taxa) are linked with poor patient outcomes as GVHD-related mortality, and insisted on the importance of the interaction between the microbiota and its host, and the opportunity to restore integrity of the intestinal microbiota (Malard et al., 2018; Taur et al., 2012; Taur et al., 2014; Peled et al, 2020; Holler et al., 2014; Golob et al., 2017; Jenq et al., 2015).

### FMT to Restore Gut Microbiota in Allo-HSCT Patients

Thus, strategies to manipulate the gut microbiota to suppress or decrease treatment-related complications in allo-HSCT patients were recently proposed, in addition to the standard of care armamentarium. Several case studies reported promising results of the use of Fecal Microbiota Transfer (FMT, also known as fecal microbiotherapy, defined as the administration of treated faeces from healthy donors via the upper or lower gastrointestinal route with the aim of restoring gut microbiota homeostasis) in the treatment of gastrointestinal aGVHD (Kakihana et al., 2016; Spindelboeck et al., 2017; Qi et al., 2018 ; van Lier et al., 2019; Shouval et al., 2018).

However, given the small number of cases reported so far in this poor prognosis population of patients, it is difficult to identify which patient should and could actually benefit from such a FMT.

In absence of a definition of the targeted population, a patient could get the FMT although his/her clinical status will not allow the achievement of clinical response or his/her microbial intestine is not prepared to benefit from the fecal transplant product.

GVHD patient population is very fragile and with high mortality rate if an effective treatment is not put in place very quickly. Thus, treating patients who are not prepared to benefit from FMT treatments may lead to a loss of time and opportunity to be treated, within the context of critical illness and life-threatening emergency.

The present invention aims at fulfilling the unmet need for a FMT performance prediction test to guide and optimize therapeutic management of GVHD patients.

### SUMMARY OF THE INVENTION

The present invention pertains to a method for assessing whether a subject in need of a complementation of his/her gastrointestinal microbiota with live bacteria (e.g., FMT) can benefit from said complementation, i.e., whether the administered live bacteria will be able to engraft, thus leading to a significant change in the composition of the subject’s gastrointestinal microbiota. This method is particularly advantageous for optimizing therapeutic management of GVHD patients, by distinguishing patients who can successfully receive such a complementation from those who will most likely not benefit from this treatment and need a conditioning treatment prior to receiving the live bacteria to improve the likelihood that these bacteria will engraft in their gut.

This method comprises:
a. measuring, in a gastrointestinal biological sample from said subject, the abundance of bacteria associated with a good prognosis, wherein said bacteria belong to at least one category selected from the group consisting of:
   Firmicutes phylum;
   Bacilli and Actinobacteria classes;
   Bacillales, Lactobacillales and Micrococcales orders;
   Staphylococcaceae, Lactobacillaceae, and Micrococcaceae families; and
   Staphylococcus, Lactobacillus, Melissococcus and Arthrobacter genera; and/or
b. measuring, in a gastrointestinal biological sample from said subject, the abundance of bacteria associated with a bad prognosis, wherein said bacteria belong to at least one category selected from the group consisting of:
   Bacteroidetes and Proteobacteria phyla;
   Bacteroidia class;
   Bacteroidales and Enterobacteriales orders;
   Bacteroidaceae, Porphyromonadaceae, Acidaminococcaceae, Lachnospiraceae, Ruminococcaceae, Clostridiaceae, Prevotellaceae and Erysipelotrichaceae families; and
   Bacteroides, Escherichia, Shigella, Ruminococcus, Faecalibacterium, Dorea, Coprococcus, Blautia, Alistipes, Subdoligranulum, Roseburia, Parabacteroides and Lachnospira genera; and
c. using the results obtained in step a and/or in step b and a calculation formula, calculating at least one score (#R) reflecting the likelihood that the subject’s microbiota be significantly improved by said complementation; and
d. comparing each score obtained in step c to one or several reference values, and deducing whether the subject can successfully receive the complementation of his/her gastrointestinal microbiota with live bacteria or whether the subject needs a preparation treatment prior to said complementation.

According to other aspects of the invention, host parameters can be combined to the above microbiota parameters to predict the success or failure of FMT or other complementation treatment with live bacteria.

The present invention also pertains to the use of a FMT product for treating GvHD in a subject for whom the test was positive.

Another aspect of the invention is the use of conditioning treatments such as non-absorbable antibiotics targeting unfavorable bacteria and/or osmotic laxative treatments, to prepare the patient so that he/she can then benefit from FMT or another complementation treatment with live bacteria.

Materials and kits for performing the method according to the invention are also provided.

### LEGENDS TO THE FIGURES

FIG. 1: clinical pathway according to the invention

FIG. 2: HERACLES study design

FIG. 3: HERACLES study, SR-aGvHD patients, BrayCurtis similarity vs IMP, OTU level

FIG. 4: HERACLES study, SR-aGvHD patients, Evolution of the similarity with product compared to V1

FIG. 5: HERACLES study, SR-aGvHD patients, MaaT indexes. Upper panel: Butycore; Middle panel: Core microbiota; Lower panel: Health MaaT index.

FIG. 6: EAP patients, BrayCurtis similarity vs IMP, OTU level

FIG. 7: EAP patients, Butycore MaaT index

FIG. 8: EAP patients, Health MaaT index

FIG. 9: Blood citrulline

FIG. 10: Indoxylsulfate

FIG. 11: fecal zonulin

FIG. 12: pre-albumin (blood)

FIG. 13: total cholesterol

FIG. 14: microbiota biomarkers of the gastrointestinal response at baseline

FIG. 15: discriminant analysis results. A: effect size for each pre-selected taxon which has a significative stratifying effect. B: taxa grouped by taxonomic levels ( P: Phylum, C: Class, O: Order, F: Family, G: Genus). Taxa with no significant effect on the prognostic signature are indicated in white.

FIG. 16: additional microbiota biomarkers of the gastrointestinal response at baseline, with thresholds (abundances measured by 16S sequencing).

FIG. 17: overall predictive analysis results. The Ridge logistic regression with internal cross-validation average AUC is illustrated as a grey line surrounded by confidence intervals (light grey ribbon) for each time point. The number of patients included at each visit is also mentioned.

FIG. 18: main markers predictive results. Each marker (rows) is considered as an important driver (quantified as weight) in the predictive model for at least one time point. The bars represent the marker corresponding weight with confidence intervals. Negative weights (bars oriented to the left) denote more important measurement values for non-responders, positive weights (bars oriented to the right) denote more important measurement values for responders.

### DETAILED DESCRIPTION

The invention is particularly relevant in the clinical context of GVHD, especially in the context of steroid-refractory GVHD (SR-aGvHD). It is an optimization of the FMT treatment. Clinicians know that:
on the one hand, treating a patient with a FMT that will poorly colonize the patient is not likely to be as efficient as wished. It can take time particularly precious for this patient population, and cost money although there is a low likelihood of treatment benefit. Indeed, some patients may need additional treatments prior to receiving the FMT, to increase the likelihood that the FMT efficiently modulates their microbiota and has clinical benefit.
on the other hand, providing such “preparing treatments” to every patient would lead to treat patients who do not need such a treatment. This would at best lead to a loss of time, and at worse decrease the likelihood that these patients respond.

Hence, the present invention aims at providing a FMT performance prediction test to distinguish the patients who need a treatment prior to FMT to increase their chance of responding thereto, from those who do not need any such preparing treatment and can directly receive the FMT with a high chance of success.

In this context, two programs were set up by the Applicant to investigate the potential benefit of FMT in the GVHD population. The results of these programs, described in the experimental part which follows, led to a stratification tool to identify patients eligible to FMT.

The present invention thus pertains to a method for assessing whether a subject in need of a complementation of his/her gastrointestinal microbiota with live microorganisms can benefit from said complementation, comprising:
a. measuring, in a gastrointestinal biological sample from said subject, the abundance of bacteria associated with a good prognosis, wherein said bacteria belong to at least one category selected from the group consisting of:
   Firmicutes phylum;
   Bacilli and Actinobacteria classes;
   Bacillales, Lactobacillales and Micrococcales orders;
   Staphylococcaceae,, Lactobacillaceae, and Micrococcaceae families; and
   Staphylococcus, Lactobacillus, Melissococcus and Arthrobacter genera; and/or
b. measuring, in a gastrointestinal biological sample from said subject, the abundance of bacteria associated with a bad prognosis, wherein said bacteria belong to at least one category selected from the group consisting of:
   Bacteroidetes and Proteobacteria phyla;
   Bacteroidia class;
   Bacteroidales and Enterobacteriales orders;
   Bacteroidaceae, Porphyromonadaceae, Acidaminococcaceae, Lachnospiraceae, Ruminococcaceae, Clostridiaceae, Prevotellaceae and Erysipelotrichaceae families; and
   Bacteroides, Escherichia, Shigella, Ruminococcus, Faecalibacterium, Dorea, Coprococcus, Blautia, Alistipes, Subdoligranulum, Roseburia, Parabacteroides and Lachnospira genera; and
c. using the results obtained in step a and/or in step b and a calculation formula, calculating at least one score (#R) reflecting the likelihood that the subject’s microbiota be significantly improved by said complementation; and
d. comparing each score obtained in step c to one or several reference values, and deducing whether the subject can successfully receive the complementation of his/her gastrointestinal microbiota with live microorganisms or whether the subject needs a preparation treatment prior to said complementation.

As used herein, the term “comprise” or “include” is intended to mean that the compositions and methods include elements selected from the recited lists, without excluding other elements.

The singular forms “a” and “the” include plural references unless the context clearly dictates otherwise. Thus, e.g., reference to “a calculation formula” includes a plurality of calculation formulas.

In the present text, a complementation of gastrointestinal microbiota with live microorganisms (e.g., a FMT) is considered as “successful” if it results in a significant change in the microbiota composition of the subject who has received this complementation. As explained in the experimental part below, the success of the complementation can be assessed using a variety of indexes, such as the OTU BrayCurtis similarity with the administered microorganisms composition (e.g., FMT product), the Butycore, Core microbiome and Health index (defined in the experimental part), as well as parameters such as blood citrullin and blood Indoxyl-3-sulfate concentrations. In particular, one can consider that the complementation of gastrointestinal microbiota with live microorganisms is successful if the OTU BrayCurtis similarity between the subject’s gastrointestinal microbiota and the product administered for such complementation increases by at least 5% a few days (e.g., 5 to 12 days) after one, 2 or 3 administrations (compared to the similarity between subject’s gastrointestinal microbiota and the product observed before the first administration of the product).

In the above method, a subject who “can successfully receive” a complementation treatment (i.e., a product comprising live microorganisms to complement his/her gastrointestinal microbiota) thus is a person for whom the complementation will likely be successful, i.e., will result in a significant change in his/her gastrointestinal microbiota composition, without needing any preparation or conditioning treatment prior to the administration of the complementation composition.

Likewise, a subject who “can benefit from” or is “likely to benefit from” a complementation treatment is a person for whom the complementation will likely be successful, i.e., will result in a significant change in his/her gastrointestinal microbiota composition without needing any preparation or conditioning treatment prior to the administration of the complementation composition.

Hopefully, a successful complementation will lead to a clinical response of the subject in need of such complementation. This is however the case only when the complementation product has been adequately chosen to improve the subject’s condition. Otherwise, the complementation can be successful as such (i.e., the administered microorganisms engraft in the subject’s gut) without effect on the subject’s health or, in the worse case, with deleterious effects. Thus, in the present text the “success of the complementation” is not synonymous with a clinical response to this treatment.

As used herein, the term “good prognosis” means prognosis that the complementation will result in the engraftment of at least part of the administered live bacteria, whereas a “bad prognosis” means prognosis that the complementation will not result in a significant change in the microbiota composition.

When performing the claimed method, the skilled person can normalize the measured abundances, using any appropriate reference. Non-limiting examples of appropriate references include the total number of bacteria, the total number of bacteria + archea and, especially when the calculation in step c is a ratio between the levels of “good prognosis” and “bad prognosis” bacteria, any internal reference.

According to a particular embodiment of the above method, the following values are determined:
a first value (#G) is determined in step a, and/or
a second value (#B) is determined in step b,
in step c, #R1=#G, #R2=1:#B and/or #R3=#G:#B, so that #R1, #R2 and/or #R3 above reference value(s) are indicative of a good prognosis and #R1, #R2 and/or #R3 inferior to reference value(s) are indicative of a bad prognosis.

In the above embodiment, #G is calculated with the measured abundances of the taxa selected for the bacteria associated with a good prognosis. For example, it can be the sum of the relative abundance of these taxa. Of course, this sum can be a weighted sum, to reflect each taxon’s importance in the prognosis. For example, the skilled person can attribute a bigger weight to a taxon usually present in very low quantities but highly relevant for the prognosis, than the weight attributed to a taxon present in large quantities but poorly relevant in the prognosis.

The same reasoning applies to the calculation of #B.

The skilled person can also use, in step c, a formula more complex than the indicated ratios. The only condition is that the reference value(s) be adapted accordingly. Of course, calculation formulas leading to null or infinite results will be precluded.

As used herein the “complementation of the subject’s gastrointestinal microbiota with live microorganisms” designates administration of any composition comprising live microorganisms, with the aim to improve the subject’s microbiota. Such a composition can comprise a pure culture of one single strain, a mix of several cultured strains and/or a complex community of microorganisms, e.g., originating from fecal material from one or several donors. Fecal Microbiota Transplantation (FMT) is an example of complementation with live microorganisms according to the invention.

According to a particular embodiment, the method according to the invention is performed for assessing whether a subject in need of a complementation of his/her gastrointestinal microbiota with live microorganisms, for example through fecal microbiota transplant (FMT), can benefit from said transplant.

According to another particular embodiment, the subject suffers from a graft versus host disease (GvHD) following allogeneic hematopoietic stem cell transplantation (allo-HSCT).

The above method is particularly useful for assessing whether a subject who suffers from an acute, steroid-refractory graft versus host disease (SR-aGvHD) following allogeneic hematopoietic stem cell transplantation (allo-HSCT) can benefit from a complementation with live microorganisms (such as FMT), and/or in situations where the subject suffers from a GvHD with gastrointestinal impact.

When performing the above method, the skilled person is free to choose any combination of taxa amongst the different taxa indicated above as associated with good or bad prognosis. Taxa of same or different taxonomic levels can be combined. The skilled person can also combine these taxa with additional ones and, as already mentioned, the skilled person can use any relevant formula to calculate #G and/or #B values, respectively associated with good and bad prognosis. Non-limitative examples of formulas for performing the invention are indicated below (n.b.: the sums indicated below are to be understood as weighted sums of the indicated taxa - the skilled person can define the weight of each taxon to optimize the predictive value of the result):
(i) #G= Firmicutes, #B= Bacteroidetes and #R=#G:#B; and/or
(ii) #G= Firmicutes phylum excluding Acidaminococcaceae and Lachnospiraceae families, #B= Bacteroidetes and #R=#G:#B; and/or
(iii) #G= Firmicutes + Actinobacteria, #B= Bacteroidetes and #R=#G:#B; and/or
(iv) #G= Actinobacteria + Firmicutes excluding Acidaminococcaceae and Lachnospiraceae families, #B= Bacteroidetes and #R=#G:#B; and/or
(v) #G= Firmicutes, #B= Bacteroidetes + Proteobacteria and #R=#G:#B; and/or
(vi) #G= Firmicutes phylum excluding Acidaminococcaceae and Lachnospiraceae families, #B= Bacteroidetes + Proteobacteria and #R=#G:#B; and/or
(vii) #G= Firmicutes + Actinobacteria, #B= Bacteroidetes + Proteobacteria and #R=#G:#B; and/or
(viii) #G= Actinobacteria + Firmicutes excluding Acidaminococcaceae and Lachnospiraceae families, #B= Bacteroidetes + Proteobacteria and #R=#G:#B; and/or
(ix) #G= Firmicutes and #R=#G; and/or
(x) #G= Firmicutes phylum excluding Acidaminococcaceae and Lachnospiraceae families, and #R=#G; and/or
(xi) #G= Firmicutes + Actinobacteria and #R=#G; and/or
(xii) #G= Actinobacteria + Firmicutes excluding Acidaminococcaceae and Lachnospiraceae families and #R=#G; and/or
(xiii) #B= Bacteroidetes and #R=1:#B; and/or
(xiv) #B= Bacteroidetes + Proteobacteria and #R=1:#B; and/or
(xv) #G= Bacilli + optionally Actinobacteria, #B= Bacteroidia + optionally Gammaproteobacteria + optionally Negavicutes + optionally Clostridia and #R=#G:#B; and/or
(xvi) #G= Bacillales + Lactobacillales + Micrococcales, #B= Bacteroidales + Enterobacteriales + optionally Selenomonadales + optionally Clostridiales and #R=#G:#B; and/or
(xvii) #G= Staphylococcaceae, + Lactobacillaceae + Micrococcaceae + optionally Enterococcaceae, #B= Bacteroidaceae + Porphyromonadaceae + Acidaminococcaceae + Lachnospiraceae + optionally Enterobacteriaceae and #R=#G:#B; and/or
(xviii) #G= Staphylococcus + Lactobacillus + Melissococcus + Arthrobacter, #B= Bacteroides + Escherichia + Shigella and #R=#G:#B.
(xix) #G= Bacilli + Micrococcales, #B= Bacteroidia + Enterobacteriales + Acidaminococcaceae + Lachnospiraceae and #R=#G:#B.
(xx) #B= Lachnospiraceae + Ruminococcaceae + Clostridiaceae, Prevotellaceae + Erysipelotrichaceae and #R=1:#B.
(xxi) #B= Bacteroides + Ruminococcus + Faecalibacterium + Dorea + Coprococcus + Blautia + Alistipes + Subdoligranulum + Roseburia + Parabacteroides + Lachnospira and #R=1:#B.

To perform the method of the invention, the skilled person can use any appropriate method for quantifying the bacteria. Non-limitative examples of such methods include quantitative PCR (qPCR), 16S sequencing, whole metagenomics sequencing, microarray, immune-detection (e.g. ELISA tests), metabolomics (e.g. Liquid Chromatography coupled to tandem Mass Spectrometry or Gas Chromatography coupled to tandem Mass Spectrometry) as well as culture and/or flow cytometry methods.

According to a particular embodiment of the invention, the abundances of the relevant taxa are measured by quantitative PCR. PCR techniques are well known and easily available and do not need a precise description. The PCR-based techniques are performed with amplification primers designed to be specific for the targets which are measured. The present invention hence also pertains to a set of primers suitable for performing the above method, i.e., a set of primers comprising primer pairs for amplifying sequences specific for each of the microorganism taxa to be detected in steps a and/or b of said method. Such a set of primers comprises a minimum of 4 primers, but it can comprise more primers, for example 6, 8, 10, 16, 20, 30, 40, 50, 60, 70, 80, 100, 200, 300, 500, 1000 or more primers. A kit of parts comprising such a set of primers and reactants for extracting bacterial DNA from a sample such as a rectal swab or stool sample is also part of the invention.

In another particular embodiment, the relative abundance of the selected species is assessed in step a and/or b by the use of a nucleic microarray. A “nucleic microarray” consists of different nucleic acid probes that are attached to a solid support, which can be a microchip, a glass slide or a microsphere-sized bead. Probes can be nucleic acids such as cDNAs (“cDNA microarray”) or oligonucleotides (“oligonucleotide microarray”), and the oligonucleotides may be about 25 to about 60 base pairs or less in length. To determine the copy number of a target nucleic acid in a sample, this sample is labelled and contacted with the microarray in hybridization conditions so that complexes form between probe sequences attached to the microarray surface and target nucleic acids that are complementary thereto. The presence of labelled hybridized complexes is then detected. Many variants of the microarray hybridization technology are available to the skilled person.

A nucleic acid microarray designed to perform the method according to the invention is hence also part of the present invention. Such a nucleic acid microarray comprises nucleic acid probes specific for each of the bacterial taxa to be detected in step a and/or b of said method. The microarray according to the invention may further comprise at least one oligonucleotide for detecting at least one gene of at least one control bacterial species and/or any spiked-in control sequence. Preferably, the oligonucleotides are about 50 bases in length. Suitable microarray oligonucleotides may be designed, based on the genomic sequences specific for the relevant taxa, using any method of microarray oligonucleotide design known in the art. In particular, any available software developed for the design of microarray oligonucleotides may be used, such as, for instance, the OligoArray software, the GoArrays software, the Array Designer software, the Primer3 software, the mopo16s software or the Promide software, all known by the skilled in the art.

According to a further embodiment, determining the abundance of the relevant taxa in a sample obtained from the subject is performed using sequencing. Optionally, DNA is fragmented, for example by restriction nuclease or mechanical fragmentation prior to sequencing. Sequencing is done using any technique known in the state of the art, including sequencing by ligation, pyrosequencing, sequencing-by-synthesis, single-molecule sequencing or next-generation sequencing. Sequencing also includes PCR-Based techniques, such as for example emulsion PCR. A number of platforms are available for performing next-generation sequencing (NGS, also called “massive parallel DNA sequencing” or “high throughput DNA sequencing”), such as, but not limited to the Illumina Genome Analyzer platform, the Roche 454 platform, the ABI SOLiD platform, the Helicos single molecule sequencing platform, real-time sequencing using single polymerase molecules (Eid et al., 2009), Ion Torrent sequencing (WO 2010/008480), PacBio sequencing (Rhoads et al., 2015) and Oxford Nanopore sequencing (Clarke et al., 2009).

According to yet another embodiment, the abundance of the relevant taxa in a sample obtained from the subject is measured through bacterial cultivation on selective media. For example, the fecal sample is diluted and then cultured under anaerobic conditions on a Petri dish with a medium selective for Firmicutes, and on another Petri dish with a medium selective for Bacteroidetes; bacteria are allowed to grow and then the colonies are counted to evaluate of the relative quantities of the 2 phyla.

The abundance of the relevant taxa in a sample obtained from the subject can also be measured by flow cytometry: for example, Firmicutes from a sample can be labeled with a fluorophore and Bacteroidetes with another fluorophore. The number of cells belonging to Firmicutes and Bacteroidetes is then assessed using a cytometer to measure the emitted fluorescence.

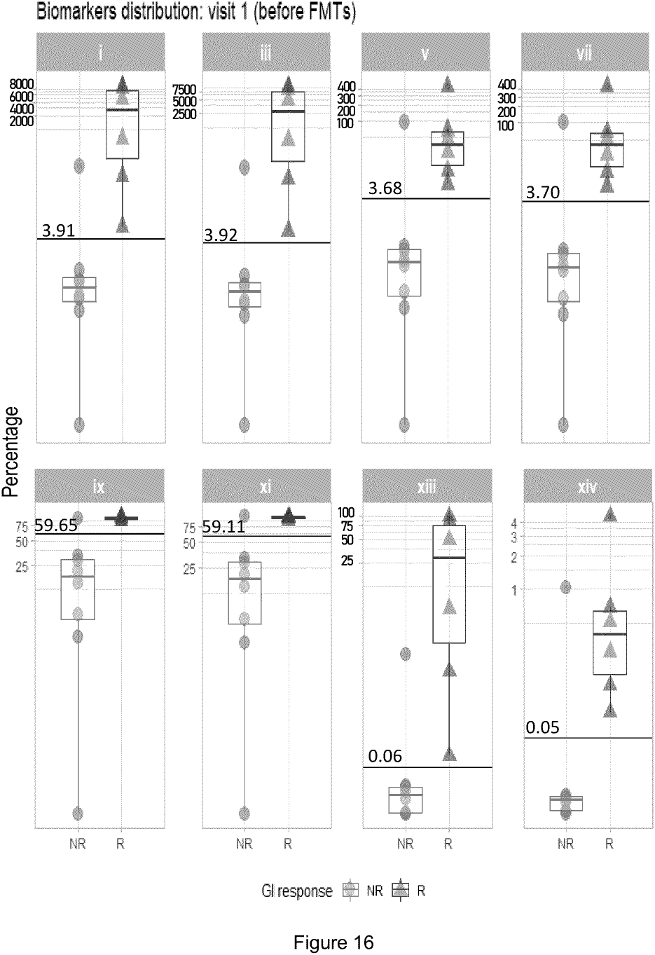

Examples of values that can be used as “reference values” in the frame of the invention are disclosed in the experimental part below (Example 5 and FIG. 16). These values were obtained from a specific cohort, with bacterial abundances measured via 16S sequencing. Other examples of reference values are as follows:
Using formula (xx) above, i.e., #B= Lachnospiraceae + Ruminococcaceae + Clostridiaceae + Prevotellaceae + Erysipelotrichaceae and #R=1:#B, a reference value is about 100, which means that #R>100 indicates that the subject is likely to benefit from the FMT.
Using formula (xxi) above, i.e., #B= Bacteroides + Ruminococcus + Faecalibacterium + Dorea + Coprococcus + Blautia + Alistipes + Subdoligranulum + Roseburia + Parabacteroides + Lachnospira and #R=1:#B, a reference value is about 50, which means that #R>50 indicates that the subject is likely to benefit from the FMT.

Of course, the skilled artisan can adapt or refine these thresholds, depending on the technique used to measure the relative abundance of the microorganisms (for example, quantitative PCR, hybridization on a microarray or sequencing), the specific condition of the patient, the nature of the GI microbiota complementation with live microorganisms (e.g., FMT) to be administered, the nature of the sample used, the patient’s food habits and other possible factors. More generally, the reference value to be considered when performing the above method is predetermined by measuring the relative abundance of the recited bacterial taxa in a representative cohort of individuals with a given condition, and whose response to a given treatment by GI microbiota complementation is known. The skilled person can also adjust the reference value(s) to favor the sensitivity and/or the specificity of the test.

According to a particular embodiment of the invention, the biological sample used in step a and/or b is a rectal swab or a feces sample.

Another aspect of the present invention relates to the prognostic value of certain host parameters (i.e., parameters distinct from the microbiota composition) for assessing whether a subject in need of a complementation of his/her gastrointestinal microbiota with live microorganisms can benefit from said complementation. This aspect is supported by the results disclosed in Example 4 below, which show the prognostic relevance of the concentration of fecal zonulin and the blood concentrations of citrullin, prealbumin, cholesterol and indoxylsulfate. Two biomarkers, the suppressor of tumorigenicity-2 (ST2) and the regenerating-islet-derived protein 3-α (REG3α), also known as MAGIC biomarkers, were previously described to predict long-term outcomes in steroid-resistant GVHD (non relapse mortality and overall survival) (Major-Monfried et al., 2018). Preliminary results from the inventors with ST2 show that these markers at baseline are also predictive of the patient’s response to FMT (data not shown).

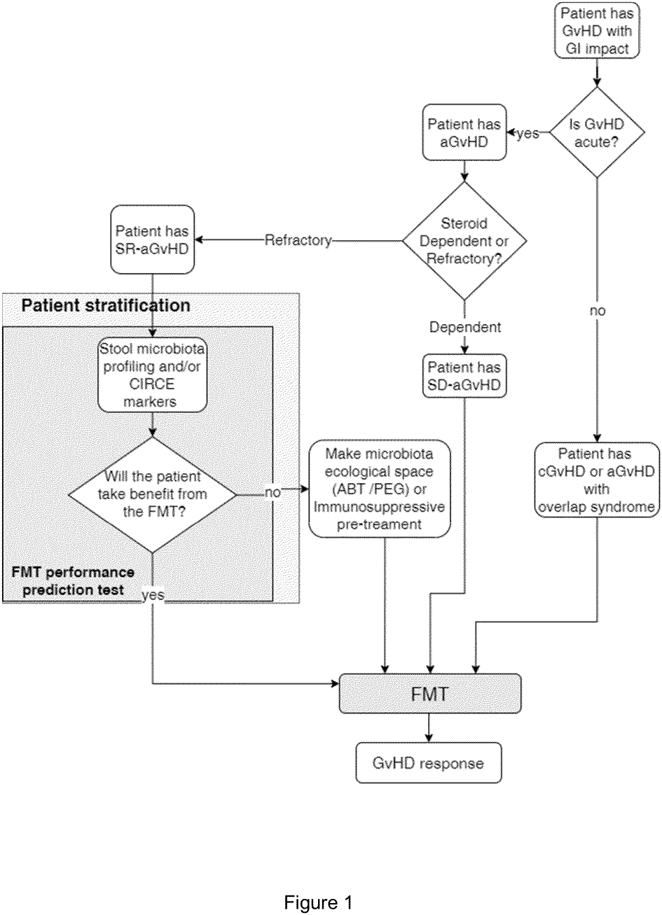

The present invention thus also relates to a method for assessing whether a subject in need of a complementation of his/her gastrointestinal microbiota with live microorganisms can benefit from said complementation, comprising:
A. from at least one biological sample from the subject, measuring one, two, three, four, five, six or seven prognostic markers selected from the group consisting of the concentrations of cholesterol, indoxylsulfate, zonulin, citrullin, prealbumin, suppressor of tumorigenicity-2 (ST2) and regenerating-islet-derived protein 3-α (REG3α); these markers are called “CIRCE markers” in FIG. 1.
B. comparing the values obtained in step a to reference values, wherein:
   fecal zonulin concentration superior to a reference value;
   citrullin concentration superior to a reference value;
   prealbumin concentration superior to a reference value;
   cholesterol concentration superior to a reference value; and/or
   3-indoxylsulfate concentration inferior to a reference value;
   ST2 concentration inferior to a reference value; and/or
   REG3α concentration inferior to a reference value; are indicators of good prognosis.

As shown in Example 7 below, the inventors also identified IL-6, IL-1β, IFNγ, CCL28, IL-8, IL-2, CCL25 and MCP_1 as additional biomarkers correlated with the success of an FMT. In particular, the levels of IL-6, IL-1β, IFNγ, CCL28 and IL-2 before the FMT are correlated with the success of said FMT.

The present invention thus also relates to a method for assessing whether a subject in need of a complementation of his/her gastrointestinal microbiota with live microorganisms can benefit from said complementation, comprising:
A. from at least one biological sample from the subject, measuring one or several prognostic markers selected from the group consisting of the concentrations of cholesterol, 3-indoxylsulfate, fecal zonulin, citrullin, prealbumin, suppressor of tumorigenicity-2 (ST2), regenerating-islet-derived protein 3-α (REG3α), IL-6, IL-1β, IFNγ, CCL28 and IL-2;
B. comparing the values obtained in step a to reference values, wherein:
   fecal zonulin concentration superior to a reference value;
   citrullin concentration superior to a reference value;
   prealbumin concentration superior to a reference value;
   cholesterol concentration superior to a reference value;
   3-indoxylsulfate concentration inferior to a reference value;
   ST2 concentration inferior to a reference value;
   REG3α concentration inferior to a reference value;
   IL-6 concentration inferior to a reference value;
   IL-1β concentration inferior to a reference value;
   IFNγ concentration inferior to a reference value;
   CCL28 concentration superior to a reference value; and/or
   IL-2 concentration inferior to a reference value; are indicators of good prognosis.

The concentration of zonulin may be measured in any appropriate sample. According to a particular embodiment of this method, fecal zonulin concentration is measured in a rectal swab or a feces sample.

In the above method, citrullin, prealbumin, cholesterol, indoxylsulfate, ST2, REG3α, IL-6, IL-2, IL-1β, IFNγ and/or CCL28, can be measured from any appropriate biological sample from the patient. Non-limitative examples of suitable biological samples include blood, serum and plasma.

Of course, the skilled person can advantageously combine the methods described above, respectively based on the analysis of the subject’s microbiota and on the analysis of certain host parameters, to increase the performance of the test. Methods combining both of these aspects are of course part of the present invention.

The present invention also pertains to the use of a composition of live microorganisms, preferably a FMT product, for treating GvHD in a subject for whom, based on the clinical patient profile and/or the result of a prediction test as above-described, the FMT is likely to succeed.

The prediction test is preferably used for at least SR-aGVHD patients. In addition, it can advantageously be applied in SD aGVHD, aGVHD with overlap syndrome or chronic GVHD patients, having already received at least one FMT and for whom FMT efficacy is not satisfactory, based on clinical symptoms and evaluation of FMT efficacy biomarkers (blood indoxyl sulfate, as well as Butycore Core microbiome and Health index defined in the experimental part below).

As used herein, the term “treating” refers to any reduction or amelioration of the progression, severity, risk of relapse and/or duration of the symptoms of GvHD (especially GI symptoms).

By “FMT product” is herein meant any fecal microbial composition obtained (directly or indirectly) from a stool sample from (i) the patient him/herself prior to the treatment that led to allogeneic hematopoietic stem cell transplantation (ii) healthy individual(s), (iii) individual(s) exhibiting a microbiota profile most likely to be efficient for improving the patient’s status, as well as to any such fecal microbial composition which has been enriched with one or several microbial strains. Several ways of conditioning fecal microbial material and conducting FMT have been described and are currently developed, and the skilled artisan is free to choose appropriate techniques for preparing the fecal microbial composition for use according to the invention, which can be freshly-prepared liquid, freeze-dried material or any other conditioning. Non-limitative examples of FMT products which can be used according to the present invention include FMT products described for example in WO2016/170285 or WO2019/171012, or products based on microbial culture of full or partial ecosystems containing at least 2 bacterial species. They can be administered either by enema or by the mean of a capsule for easier consumption (as described in WO2019/097030 for example), in which the product has been freeze-dried and powdered (as described in WO2017/103550 for example).

According to a particular embodiment, the subject treated by FMT according to the invention suffers from SR-aGvHD.

According to another particular embodiment, the subject treated by FMT according to the invention has gastrointestinal symptoms.

The FMT prediction test described above can be included in a broader clinical pathway for GVHD patients, described in FIG. 1, that leverages the potential of FMT to treat such diseases. According to the GVHD subcategory, the patient is either oriented directly to the FMT therapy, or the potential effect of the FMT is tested. This is the “stratification” block that is built with the “FMT performance prediction Test”. If the patient is identified by the above-described predictive test as less likely to benefit from FMT, he/she will be oriented to a treatment (e.g., antibiotherapy, PEG, ...) with the purpose to prepare his/her microbiota ecosystem beforehand the FMT. This GVHD clinical pathway (FIG. 1) is also part of the present invention.

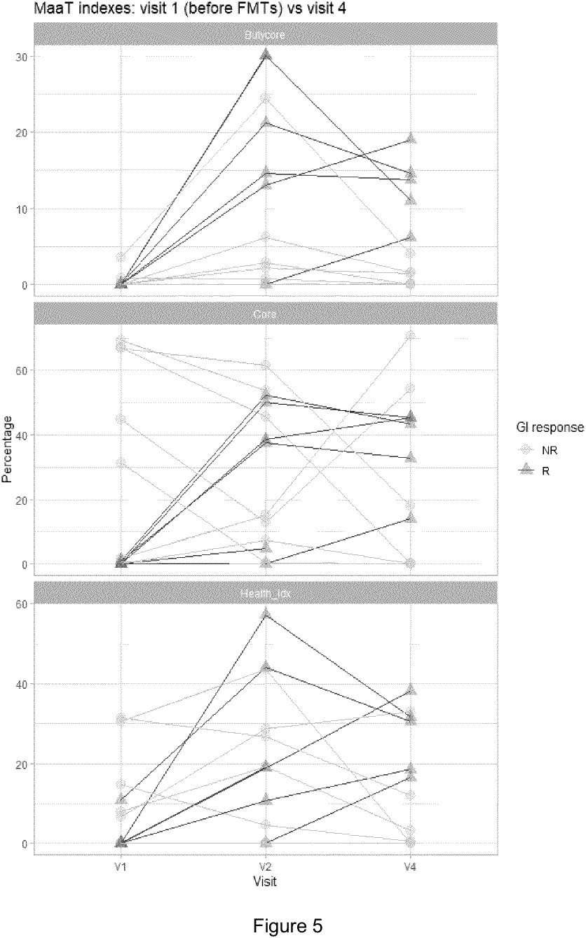

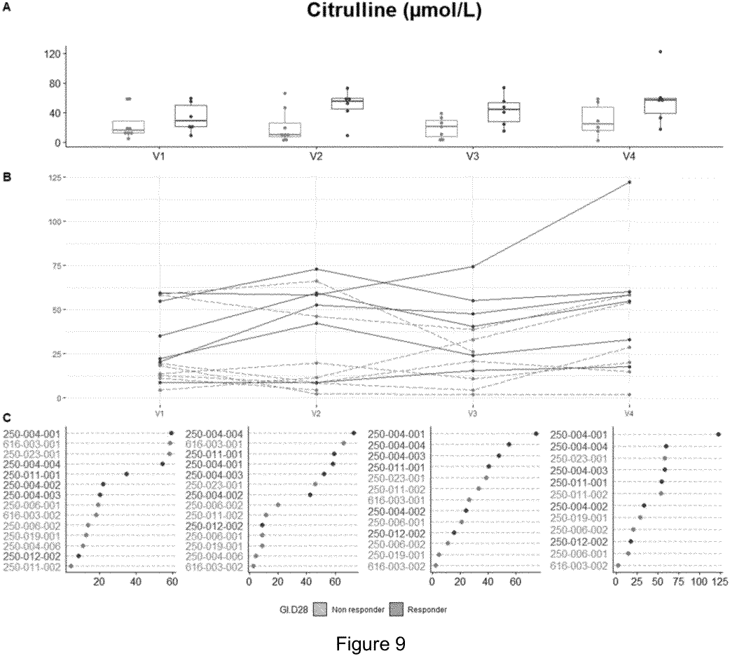

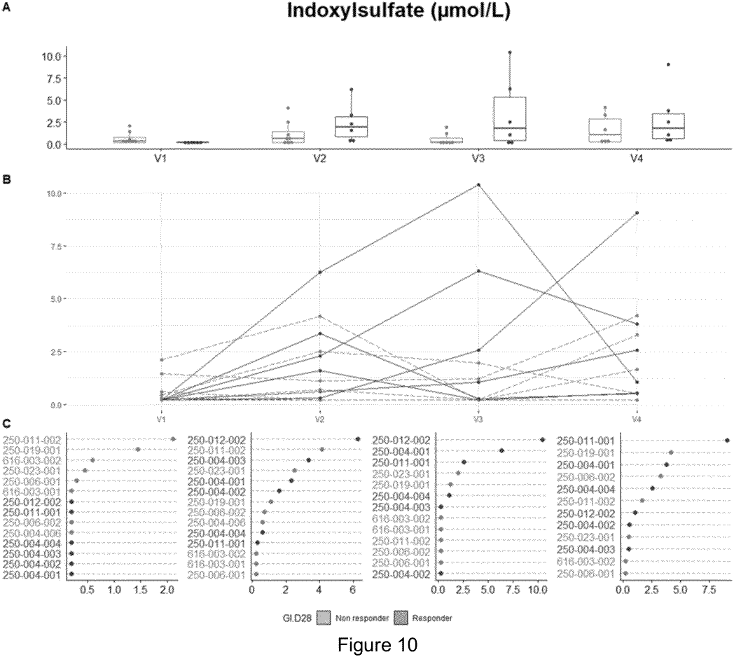

This GVHD clinical pathway can also comprise an additional step of monitoring the response to the FMT. Indeed, as shown in the experimental part below (FIG. 4), an OTU BrayCurtis similarity with the FMT product that increases by more than 5 percentage points defines a block of patients who have a good GI response. The increasing Butycore or Health index (FIG. 5) can also be used for monitoring purposes. Parameters measured in blood as citrullin (FIG. 9) and Indoxyl-3-sulfate (FIG. 10) concentrations, which increase for patients who have a good GI response as soon as the FMT occurred (visit 2), represent two alternative or additional means to monitor the response to the FMT.

The status of persons identified by the above-described predictive test as likely not to respond complementation of their gastrointestinal microbiota with live microorganisms can be significantly improved by a conditioning pre-treatment. Indeed, it is possible to induce a microbiota or host modification in patients identified as “non eligible” to FMT in order to make them eligible to FMT. For example, FMT pre-treatment with non-absorbable ABT targeting specific bacterial population, use of osmotic laxatives to reduce the burden of pathobionts in the gut, use of immunosuppressants to reduce the inflammatory state of the gut, or use of prebiotics to induce a shift in microbial communities - or any other process that addresses an ecological modification need, can be adapted to the particular patient condition. Based on the markers described above, the skilled person can identify what ecological preparation would be the best for improving the product acceptability, and subsequently increase response likelihood. This preparation is preferably designed to at least eliminate bacteria who belong to the Bacteroidetes or the Proteobacteria phyla.

Non-limitative example of FMT pre-treatments include:
non-absorbable antibiotics targeting specific bacterial population (e.g., vancomycin, gentamicin, colimycin, rifaximin, metronidazole, penicillin G and mixtures thereof),
use of osmotic laxatives to reduce the burden of pathobionts in the gut
use of prebiotics to induce a shift in microbial communities
use of immunosuppressants to reduce the inflammatory state of the gut, and
any other process that addresses an ecological modification need.

According to a particular embodiment, the present invention pertains to the use of one or several non-absorbable antibiotic(s) selected from the group consisting of vancomycin, rifaximin, metronidazole, penicillin G and mixtures thereof, for treating a GVHD patient (with or without gastrointestinal symptoms) identified as likely not to respond to FMT. More particularly, the patient suffers from SR-aGvHD. According to this aspect of the invention, the antibiotic is administered prior to a FMT (or other treatment with live microorganisms).

According to another particular aspect of the invention, the patient receives an osmotic laxative in addition to or in replacement of the non-absorbable antibiotic targeting specific bacterial population. When both the osmotic laxative and the antibiotics are administered, the antibiotics are preferably administered prior to the laxative treatment.

As already mentioned, the present invention also relates to materials such as sets of primers and nucleic acids microarrays specifically designed to perform the above-described diagnostic/prognosis methods. Kits for companion diagnostic assay, comprising such materials, are thus also part of the present invention.

Other characteristics of the invention will also become apparent in the course of the description which follows of the biological assays which have been performed in the framework of the invention and which provide it with the required experimental support, without limiting its scope.

### EXAMPLES

The present invention is supported by the results of two programs set up to investigate the potential benefit of FMT in the GVHD population:
1) The ongoing phase 2 study (HERACLES) conducted by MaaT Pharma, that investigates the efficacy of a pooled FMT biotherapeutic, MaaT013 (described in WO2019/171012). We now expect that full ecosystem gut microbiota restoration with the MaaT013 biotherapeutic could be an effective treatment of gastrointestinal predominant SR-aGVHD, and thereby reduce the risk of life-threatening complications after allogeneic HSCT.
2) An Early Access Program (EAP) allowing the treatment of any GVHD situation (chronic/ acute, SD/SR).

These programs are further described below, as well as the materials and methods used to obtain the results described herein.

### Heracles Study

The population of the study consists of patients who developed a first episode of Grade III or IV aGVHD (= gastrointestinal stages 2 to 4) with gut predominance if other organs involved, resistant to a first line therapy with steroids, aged over 18 years old.

The primary objective of this study is to evaluate the gastrointestinal response at D28 through Complete Response (CR) and Very Good Partial Response (VGPR) of steroid refractory (SR) gastro-intestinal (GI) acute graft-versus-host disease (aGVHD) patients treated with allogeneic Fecal Microbiota Transfer (FMT).

The FMT product used during this study is the MaaT013 microbiota biotherapeutics manufactured by MaaT pharma. This product was obtained as described in WO2019/171012. It is referred in the figures as “IMP” for “investigational medicinal product”.

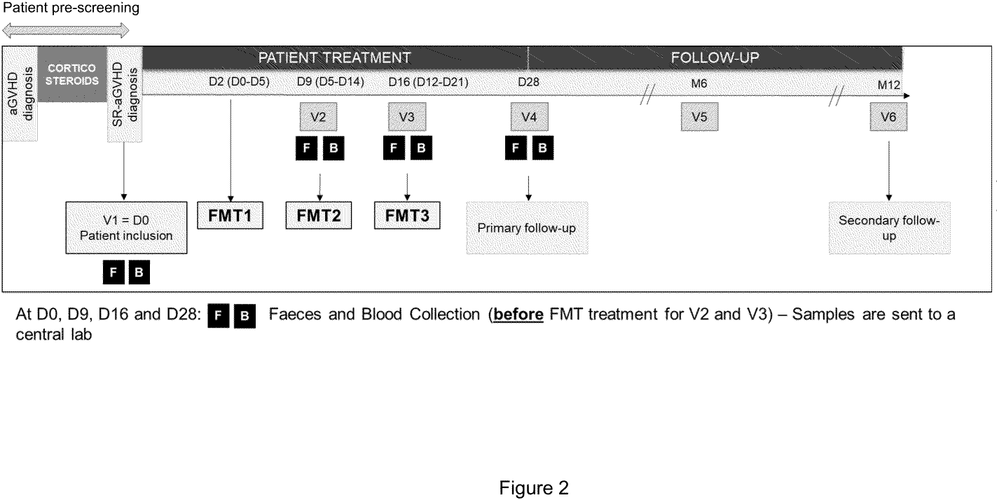

FIG. 2 shows the design of the study.

### Cohort of 15 Patients

6 responders = R
8 non-responders = NR
1 patient died before V2 (not considered in the analyses)

### Blood and Fecal Samples Were Collected at 4 Visits

V1: before FMT
V2: after FMT 1
V3: after FMT 2
V4: after FMT 3 (D28)

Some patients who failed to respond to FMT treatments received only 1 or 2 FMTs instead of 3 as planned.

The visit 1 (V1) stool collection allowed a microbiota profiling of the patient at baseline, i.e., before receiving the FMT treatment. In the below analysis, patients are separated according to their gastro-intestinal (GI) response 28 days (D28) after FMT.

The evaluation of treatment responses was automatically calculated according to the following logic, based on GVHD grading and staging performed by the physicians at V4 (Day 28). The responses were calculated compared to GVHD evaluation at baseline (V1).

GI Response was considered as achieved in the following cases:
Complete Response (CR): complete resolution of GI aGVHD manifestations, i.e. an improvement of the GI staging from any stage to 0
Very Good Partial Response (VGPR): improvement of at least 2 stages in the severity of GI aGVHD, or improvement of the GI staging from 2 to 1, except improvement to stage 0
Partial Response (PR): improvement of one stage in the severity of GI aGvHD, except improvement to stage 0 or improvement of the GI staging from 2 to 1

Patients were considered as non-responders in the following cases:
Stable Disease (SD): persistence of the same stage of GI aGvHD
Progressive Disease (PD): worsening of GI aGvHD of at least 1 stage
If the patient receives additional systemic GVHD therapy before D28 (V4)
If the patient dies before D28 (V4)

### Early Access Program (EAP)

EAP was launched to answer the growing demands from physicians to treat GVHD patients with FMT.

All types of GVHD (acute/ chronic/overlap syndrome; SR or SD) treated with steroids associated with other lines of systemic treatment could be included in the EAP program, based on physician judgement regarding the medical need.

27 patients were treated with MaaT013.
19 patients had SR-aGVHD
8 patients:
   1 patient with SR-cGVHD
   4 patients with SD-aGVHD
   2 patients with SD-aGVHD with overlap syndrome
   1 patient with SR-aGVHD with overlap syndrome

The only data available for all of these patients are clinical GVHD responses.

For 4 patients, we obtained stool samples before each FMT treatment and at D28 (1 SR-cGVHD, 2 SD-aGVHD and 1 SD-aGVHD with overlap syndrome).

GI Response was considered as achieved in the following cases, 28 days after the first MaaT013 administration:
Complete Response (CR): complete resolution of GI aGVHD manifestations, i.e., an improvement of the GI staging from any stage to 0
Very Good Partial Response (VGPR): improvement of at least 2 stages in the severity of GI aGVHD, or improvement of the GI staging from 2 to 1, except improvement to stage 0
Partial Response (PR): improvement of one stage in the severity of GI aGvHD, except improvement to stage 0 or improvement of the GI staging from 2 to 1

Patients were considered as non-responders in the following cases:
Stable Disease (SD): persistence of the same stage of GI aGvHD
Progressive Disease (PD): worsening of GI aGvHD of at least 1 stage
If the patient receives additional systemic GVHD therapy before D28
If the patient died before D28

### Material and Methods

### 16S rDNA Sequencing and Bioinformatics Analysis of Fecal Microbiota

16S rDNA sequencing was performed by Eurofins Genomics (Ebersberg, Germany). Genomic DNA was extracted using the NucleoSpin Soil kit (Machery Nagel). A sequencing library targeting the V3-V4 region of the 16S rRNA gene was constructed for each sample using the MyTaq HS-Mix 2X, Bioline, according to the manufacturer’s instructions. Libraries were then pooled in an equimolar mixture and sequenced in paired-end (2×300 bp) MiSeq V3 runs, Illumina.

After amplicon merging using FLASH (Magoč et al., 2011) and quality filtering using Trimmomatic (Bolger et al., 2014), host sequence decontamination was performed with Bowtie2 (Langmead, Ben and Salzberg, 2013). Operational Taxonomic Unit (OTU) sequence clustering was performed with an identity threshold of 97% using VSEARCH (Rognes et al., 2016) and taxonomic profiling was then performed with the Silva SSU database Release 128. For fair comparison, the sequence number of each sample was randomly normalized to the same sequencing depth i.e. 60000 amplicons per sample. Taxonomic and diversity analyses were performed with R Statistical Software (version 3.4.4).

### Host Parameters (Blood, Fecal)

Albumin, Pre-albumin, total cholesterol were assessed on plasma with Cobas Integra 400+ / Kits ALBT2, PREA, CHOL2 from Roche Diagnostics respectively.

Indoxylsulfate and citrullin were assessed on plasma by Liquid-Chromatography - Mass Spectrometry.

Fecal zonulin was assessed on stool supernatants using ELISA kit (ELx800 reader/ IDK zonulin ref K5600) from Immundiagnostik AG.

### Example 1: FMT Efficacy for Patients With Various GVHD (EAP Program)

Amongst the 19 patients with SR-aGVHD:
10 patients were considered as Non-responders to MaaT013
9 patients were considered as Responders to MaaT013.

Amongst the 8 patients with various GVHD except SR-aGVHD, all achieved GVHD response to MaaT013 (6 complete responses, 2 very good partial responses), which supports the direct orientation of these patients to a MaaT013 FMT.

### Example 2: Relationship Between Colonization Performance of the FMT Treatment and GI Response (Heracles Study)

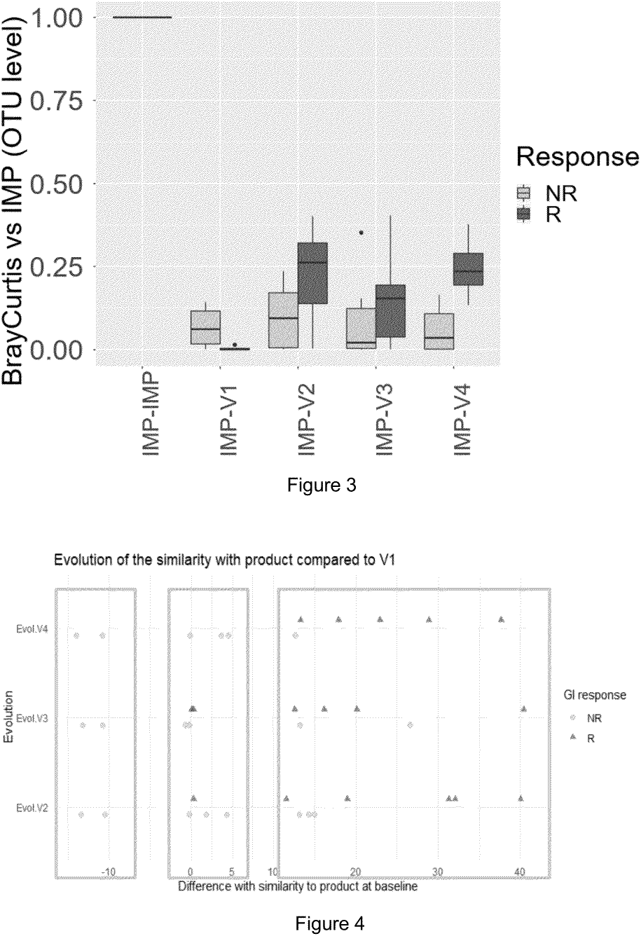

FIG. 3 depicts the patient’s microbiota similarity with the composition of the administrated FMT product, referred to as IMP. The higher the Bray Curtis value, the more similar to the product.

At V1, this similarity is low as expected because the FMT has not been administered yet. We also observe a difference between R and NR microbiotas. This may be related to the higher diversity of the NR patients’ microbiota that increases the odds of having common OTUs with the product by chance.

At V2, V3 (after FMT pass 1 and FMT pass 2, respectively), the similarity with the product composition increases only for patients considered as responders at D28.

At V4, meaning D28, after the last FMT, this difference is the most pronounced (t-test, p<0.05).

These data show that the patients’ gut microbiotas do not react equally to the FMT. Some of them, i.e., the responders, have a microbiota composition that is modified after the FMT, and these modifications lead to a composition that gets closer to the administered product; some others, mostly the non-responders, do not get more similarity with the product.

FIG. 4 illustrates the difference of the similarity percentage with the product (as described in FIG. 3) between each of the V2, V3, V4 visits and the V1 visit. The range of evolution values can be divided into 3 groups: (1) group for which the evolution is negative, (2) group for which the evolution is low [close to 0, less than 5], and (3) group for which the difference is higher than 10. At V4, all responders are this third block, which includes also one of the non-responders (the lowest value in this block).

According to these evolution results, a similarity with the product that increases by a minimum of 5 percentage points is a good candidate to define a colonization by the product that impacts the patient’s microbiota.

Conclusion: the similarity between the gut microbiota and the product microbiota is a FMT acceptance proxy, and the acceptance of the FMT is at least one of the factors that leverages patients’ response.

### MaaT Indexes

Based on the combination of public and internal data, MaaT pharma has defined 3 indexes:
Core microbiome: among public data and MaaT data for healthy subjects, it has been defined a common microbiome named core microbiome. The selected genera are the ones with >80% prevalence in the cohorts, and >0.1% median abundance per cohort. The genera list is: Ruminococcus, Faecalibacterium, Dorea, Coprococcus, Blautia, Alistipes, Bacteroides, Subdoligranulum, Roseburia, Parabacteroides, Lachnospira.
Health index: an index of a healthy microbiome (containing bacteria families often associated with good health). This index is built by summing the relative abundances from those bacteria families: Lachnospiraceae, Ruminococcaceae, Clostridiaceae, Prevotellaceae, Erysipelotrichaceae.
Butycore: sum or relative abundances of 15 butyrate producing genera: Blautia, Faecalibacterium, Alistipes, Eubacterium, Bifidobacterium, Ruminococcus, Clostridium, Coprococcus, Odoribacter, Roseburia, Holdemanella, Anaerostipes, Oscillibacter, Subdoligranulum and Butyrivibrio. The butycore can be interpreted as an anti-inflammatory potential microbiota marker.

As shown in FIG. 5, the 3 MaaT defined indexes have increased between V1 and V4 in all responder patients. The Butycore, close to 0 at V1, is greater than 5% at V4 for all responders whereas it is below 5% for all non-responders. This metric is another good candidate for assessing the quality of the colonization performance.

### Example 3: Relationship Between Colonization Performance of the FMT Treatment and GI Response (EAP Program)

For EAP patients with stool samples (1 SR-cGVHD, 2 SD-aGVHD and 1 SD-aGVHD with overlap syndrome - all were responders), according to data obtained during the EAP program, colonization performance metrics support the pattern outlined for SR-aGvHD data.

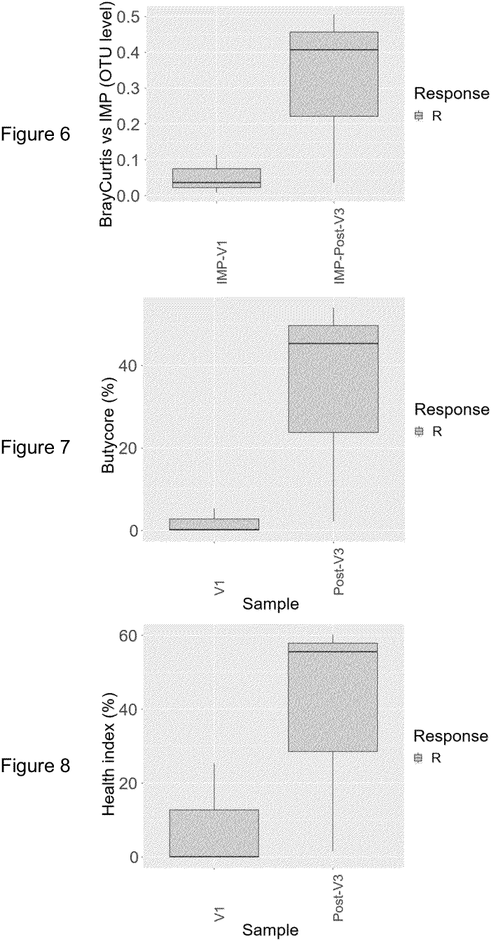

FIG. 6 depicts the similarity of patients’ microbiota with the composition of the administered IMP at V1 and Post-FMT3. The higher the Bray Curtis value, the more similar to the product. This demonstrates the engraftment of the microbiota of the IMP.

FIG. 7 depicts the Butycore measured for the IMP, for patients at V1 and Post-V3. The Butycore has an increasing pattern from V1 to Post-V3, illustrating the quality of the colonization performance and the efficiency of FMT in microbiota reconstruction.

FIG. 8 illustrates the Heath index measured for patients at V1 and Post-V3. The Heath index has an increasing pattern from V1 to Post-V3, illustrating the quality of the colonization performance and the efficiency of FMT in microbiota reconstruction.

Conclusion: These data show that the patients’ gut microbiotas do not react equally to the FMT. They have a microbiota composition that is modified after the FMT, and the modifications lead to a more diverse composition that gets closer to the administered product for those patients who are all responders.

### Example 4: Evidences for Host Parameters Markers (Heracles Study)

### Citrulline

Citrulline is an amino acid produced exclusively in small bowel enterocytes.

Because citrulline is not metabolized by the liver, its serum concentration correlates strongly with total functional enterocyte mass. It also correlates with age. Values can be influenced by renal function. Normal range of citrulline is 30-50 umol/L.

Citrullinemia is reduced in GI-aGVHD patients (Vokurka et al, Med Sci Monit 2013).

As shown in FIG. 9, citrullin levels were higher in R patients after MaaT013 dosing (significant at V2 and V3).

The baseline value of citrullin also is a predictive biomarker, citrullin > 20 µmol/L indicating that the patient is likely to respond to the treatment.

### Indoxyl Sulfate

Indoxyl sulfate is a metabolite of I-tryptophan:

I-tryptophan → indole → indoxyl → indoxyl sulfate (IS)

Indole is produced from I-tryptophan in the human intestine via tryptophanase-expressing gastrointestinal bacteria. Indoxyl is produced from indole via enzyme-mediated hydroxylation in the liver. Subsequently, indoxyl is converted into indoxyl sulfate by sulfotransferase enzymes in the liver.

Urinary 3-IS levels predict outcome after HSCT and are associated with antibiotics. Low 3-IS levels within the first 10 days after HSCT are associated with significantly higher transplant-related mortality and overall lower survival 1 year after HSCT. Not only the diversity of the microbiome but its specific composition is indicative of urinary 3-IS. The majority of OTUs associated with high urinary 3-IS levels belong to the families of Lachnospiraceae (Eubacterium rectale) and Ruminococcaceae. Low 3IS were associated with members of the class of Bacilli (Weber et al., Blood 2015).

Factors associated with 3IS: lower IS concentrations in patients receiving ciprofloxacin/metronidazole compared with patients receiving rifaximin. Earlier systemic antibiotics treatment was also associated with low 3IS levels.

A decreased urinary excretion of 3-indoxyl sulfate (3-IS) is a marker of gut microbiota disruption and increased risk of developing gastrointestinal (GI) graft-versus-host-disease (Weber et al., supra).

3-IS could not be assessed in urines of HERACLES patients (no urine collected) but we decided to test it in blood samples. Poor data are available in blood.

As shown in FIG. 10, IS levels are a bit higher in R patients at V2, V3 and V4. IS levels seem to be increased after MaaT013 dosing, suggesting a beneficial impact of MaaT013 and may be a surrogate marker of engraftment.

### Fecal Zonulin

Human zonulin is a protein that increases permeability in the epithelial layer of the small intestine by reversibly modulating the intercellular tight junctions.

Among the several potential intestinal stimuli that can trigger zonulin release, small intestinal exposure to bacteria and gluten are the two triggers that have been identified so far. Enteric infections have been implicated in the pathogenesis of several pathological conditions, including allergic, autoimmune, and inflammatory diseases, by causing impairment of the intestinal barrier. Small intestines exposed to enteric bacteria secrete zonulin. This secretion is independent of the virulence of the microorganisms tested, occurred only on the luminal aspect of the bacteria-exposed small intestinal mucosa, and is followed by an increase in intestinal permeability coincident with the disengagement of the protein zonula occludens (ZO)-1 from the tight junctional complex. This zonulin-driven opening of the paracellular pathway may represent a defensive mechanism, which flushes out microorganisms, thereby contributing to the innate immune response of the host against bacterial colonization of the small intestine (Fasano, Clin Gastroenterol Hepatol 2012).

Fecal zonulin is elevated in Crohn’s disease. Normal range is 61±46 ng/ml (Malíčková et al, Pract Lab Med 2017).

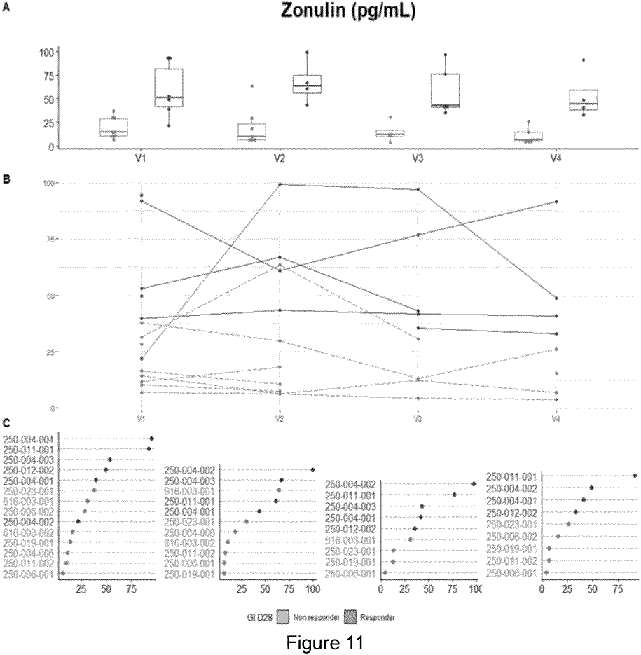

As illustrated in FIG. 11, ¾ responders measured at V2 have a slightly higher zonulin level than V1. At the cohort level, fecal zonulin is higher in responders than non-responders for all visits.

### Prealbumin

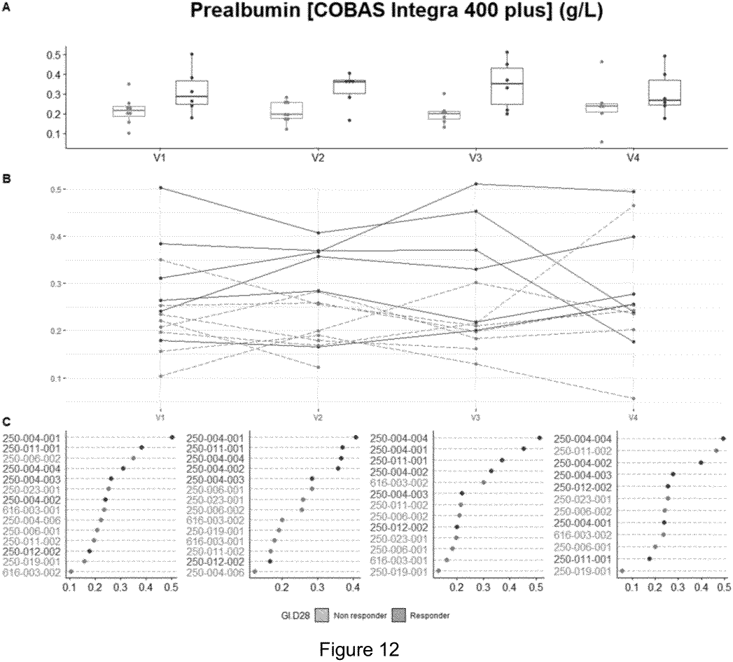

FIG. 12 illustrates that prealbumin measurements are higher in responders than non-responders, more significantly at V2 and V3.

### Total Cholesterol

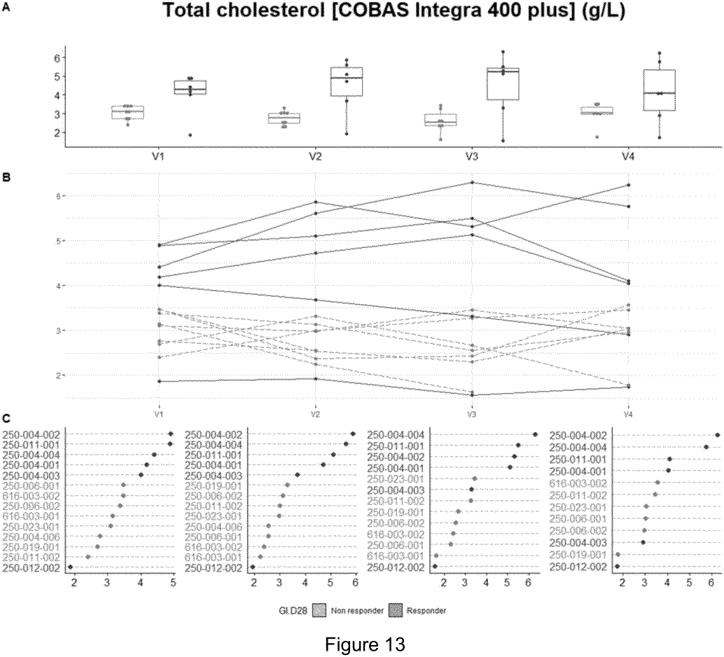

As shown in FIG. 13, cholesterol is higher in responders (all visits). Subject 250-012-002, who has the lowest value for all visits, is the exception.

### Example 5: Evidences for Microbiota Markers Predicting FMT Response

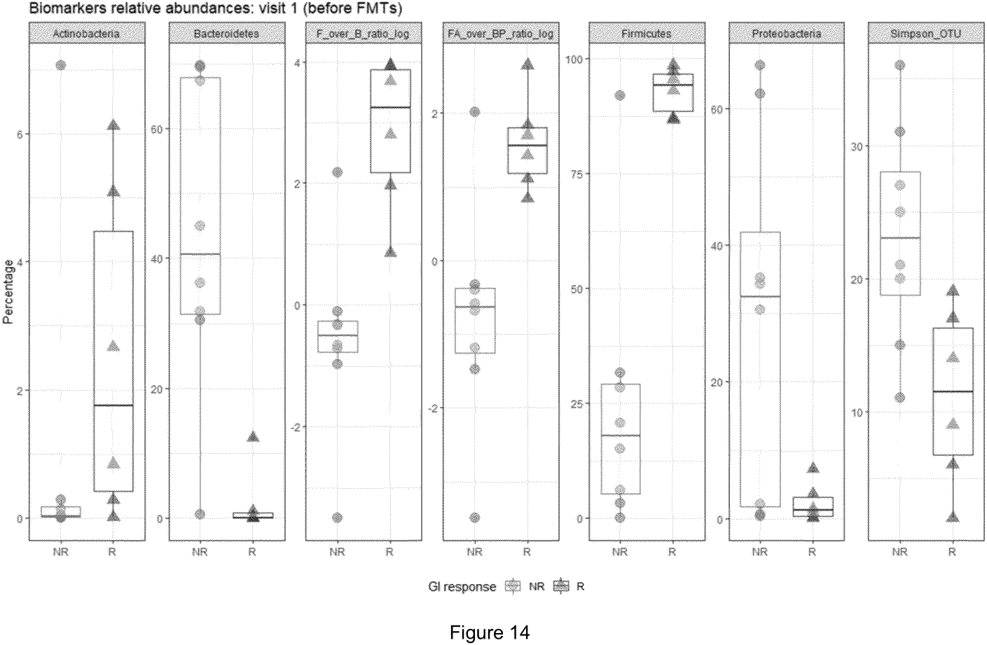

FIGS. 14 and 16 show a series of microbiota biomarkers, able to separate the population that responds (GI D28) from population that does not. The relative abundance of bacteria that belong to the Firmicutes phylum is a stratifying metric between responders (in dark grey) who have a higher relative abundance of Firmicutes (more than 80%), and non-responders (in light grey) who have a relative abundance of Firmicutes lower than 30% except for one whereas the FMT has never been administered (V1). The Actinobacteria also tend to be higher for most of responders (except for the highest value, 7%, which is reached by a non-responder). Bacteroidetes (less than 15% for R, more than 25% for most of NR) and Proteobacteria phylum (less than 10% for R, more than 25% for most of NR) have the opposite pattern: responders have lower values.

We also evaluated the ratio of good prognosis over bad prognosis phyla: (1) F_over B_ratio_log which is the Iog10 transformation of the ratio Firmicutes over Bacteroidetes, and (2) FA_over_BP_ratio_log which is the Iog10 transformation of the ratio (Firmicutes + Actinobacteria) over (Bacteroidetes + Proteobacteria). For both of these ratios, all R have a value greater than 0, and all NR except ⅛ have a lower value (FIG. 14).

Also, high alpha diversity indexes (represented here by the Simpson index at the OTU level) are likely to be bad prognosis biomarkers. All R have a Simpson index below 19%, which is not the case for 6/8 NR (FIG. 14).

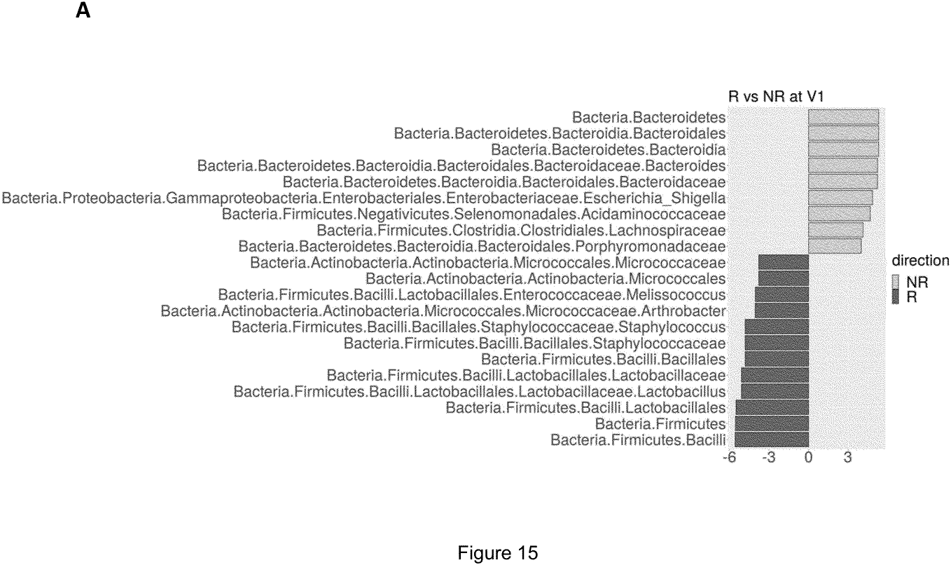

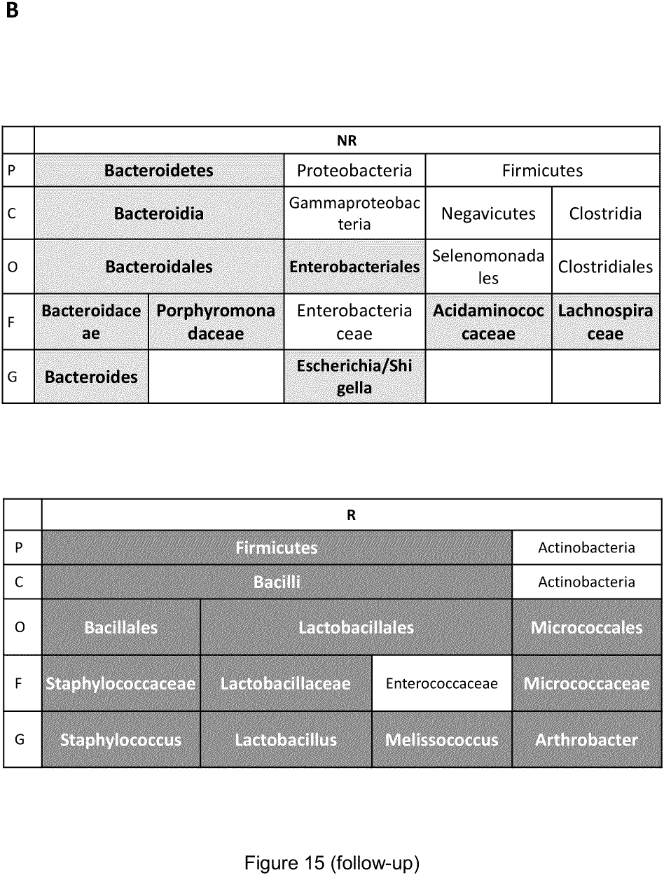

FIG. 15 depicts the result of an analysis that selects important features which are informative in the separation of several groups (here 2 groups: responders and non-responders) and measures their quantitative effects. FIG. 15A illustrates the size effect for each pre-selected taxon which has a significant stratifying effect. These taxa are presented in FIG. 15B, grouped by taxonomic levels (P: Phylum, C: Class, O: Order, F: Family, G: Genus). Colored taxa are those which are useful for patients stratification, while the other indicated taxa have no effect on stratification.

Higher relative abundances of taxa highlighted in dark grey for a given patient before the FMT is predictive of a patient response, and higher level for the ones highlighted in light grey, or as illustrated in FIG. 14, Health index, or core microbiota or diversity, is predictive of a non-response.

FIG. 16 and in Table 1 below show non-limiting examples of formulas which can be used when performing the present invention.

TABLE 1

examples of formulas and associated thresholds (#R cut-offs) for performing the FMT performance prediction test in a SR-aGvHD patient with GI symptoms, with abundances of the recited taxa measured by 16S sequencing

| Combination | #R cut-off |
|---|---|
| (i) #G= Firmicutes, #B= Bacteroidetes and #R=#G:#B; | 3,909 |
| (iii) #G= Firmicutes + Actinobacteria, #B= Bacteroidetes and #R=#G:#B; | 3,9225 |
| (v) #G= Firmicutes, #B= Bacteroidetes + Proteobacteria and #R=#G:#B; | 3,6848 |
| (vii) #G= Firmicutes + Actinobacteria, #B= Bacteroidetes + Proteobacteria and #R=#G:#B; | 3,7025 |
| (ix) #G= Firmicutes and #R=#G; | 59,651 |
| (xi) #G= Firmicutes + Actinobacteria and #R=#G; | 59,1075 |
| (xiii) #B= Bacteroidetes and #R=1:#B; | 0,057 |
| (xiv) #B= Bacteroidetes + Proteobacteria and #R=1:#B | 0,047 |

### Example 6: Relative Abundance Assessments Using qPCR

Quantitative PCR (qPCR, or real time PCR) will advantageously be used for performing the FMT performance prediction test according to the invention, for example using the protocol and primers described below.

### A. Choice of the qPCR Technology

Quantitative PCR (qPCR, or real time PCR) has several advantages because only a small amount of template DNA is required, it has a high sensitivity, a high-throughput processing, an affordable cost, and requires affordable equipment that is frequently found in laboratories (Bacchetti De Gregoris et al., 2011).

### B. Protocol Example

Note: this protocol can evolve according to new primer design with updated 16S RNA gene sequences database.

DNA is extracted using a manufactured kit suitable for the extraction of DNA from fecal material, according to the manufacturer’s instructions.

qPCR is performed in duplicates with a mix including SYBR and run on a multiwell (e.g. 96-well plate) real time PCR detection system.

Primers specific to the 16S rRNA region of bacterial taxa are used.

Each taxon-targeted qPCR (i.e., each pair of primers) has to be carried out independently.

Examples of primers which can be used are described in Table 2 below.

TABLE 2

examples of primers which can be used to measure abundances of the recited taxa by qPCR when performing the FMT performance prediction test. Nucleotide symbols: R = A or G; Y = C or T; W = A or T; and S = C or G

| Targeted taxa | Sequence (5′-3′) | SEQ ID No | Reference |
|---|---|---|---|
| Firmicutes | F: GGAGYATGTGGTTTAATTCGAAGCA R: AGCTGACGACAACCATGCAC | 1 2 | Guo et al., 2008 |
| Bacteroidetes | F: GTTTAATTCGATGATACGCGAG R: TTAASCCGACACCTCACGG | 3 4 | Yang et al., 2015 |
| Bacteria | F: AGAGTTTGATCCTGGCTCAG R: AAGGAGGTGWTCCARCC | 5 6 | Bacchetti De Gregoris et al., 2011 |

PCR cycles parameters shall be optimized for this specific assay, as well as each primer pair efficiency. An example (Yang et al., 2015) provides these values:

Material: PCR system sequence detector with 2xFastStart SYBR green mix (Vazyme, Nanjing, China). qPCR mixtures contained 10 µl of 2xFast-Start SYBR green with dye1, 0.5 µl of each forward and reverse primer (final concentration, 0.4 µM), and 9µl of the DNAtemplate (equilibrated to 10 ng).

Annealing temperature of bacterial primers: 60° C.

Cycling conditions of denaturation: 95° C. for 10 min, followed by 40 cycles of 95° C. for 15 s and 60° C. for 1 min.

Positive and negative controls shall be used.

### C. Relative Abundance Values for Bacterial Taxa

Relative abundance values for bacterial taxa can be computed (Yang et al., 2015) to total bacteria as follows:

$$\text{X=} \frac{{\left(\text{Eff} \text{.Bact}\right)}^{C {T}_{\text{bact}}}}{{\left(\text{Eff} \text{.Spec}\right)}^{C {T}_{\text{spec}}}} \times 100$$

where Eff. Bact (value between 1 and 2) is the calculated efficiency of the bacterial primers (2 = 100% and 1 = 0%), and Eff.Spec refers to the efficiency of the taxon-specific primers (Firmicutes, Bacteroidetes). CT_{bact} and CT_{spec} are the CT values registered by the thermocycler. “X” represents the percentage of 16S taxon-specific (e.g Firmicutes) copy number existing in a sample.

### Example 7: Identification of Additional Markers Predicting FMT Response

### Materials and Methods

Experiments were led on 24 patients in total. Final HERACLES cohort comprises 9 additional patients not presented in previous examples.

Using a Luminex assay, the concentrations of the following molecules were measured in plasma samples from patients included in the HERACLES study at V1, V2, V3 and V4:
CCL25 C-C Motif Chemokine Ligand 25
CCL28 C-C Motif Chemokine Ligand 28
CD14 Cluster of differentiation 14
CD30 Cluster of differentiation 30
IFN_gamma Interféron gamma
IL_10 Interleukin 10
IL_17A Interleukin 17A
IL_18 Interleukin 18
IL_1beta Interleukin 1 beta
IL_2 Interleukin-2
IL_2RA Interleukin-2 receptor alpha chain
IL-6 Interleukin 6
IL_8 Interleukin-8
IP_10 Interferon gamma-induced protein 10
MCP_1 Monocyte chemoattractant protein 1
REG3a - Regenerating islet-derived protein 3 alpha
TGFb_1 Transforming growth factor beta 1
TGFb_2 Transforming growth factor beta 2
TGFb_3 Transforming growth factor beta 3
TNFalpha Tumor necrosis factors

The Luminex assay was performed with MAGPIX® System, and the following Luminex kits : TGFb1.2.3 and sCD14 from Merck Millipore, and 16 plex Luminex from Biotechne for IL-1b, IL-2, sIL-2ra, IL-6, IL-8, IL-10, IL-17A, IL-18, IFNg, TNFa, MCP1, CCL25, CCL28, sCD30, CXCL10 and Regllla.

The concentrations of the following parameters were measured in serum samples from patients included in the HERACLES study at V1, V2, V3 and V4 with the associated methods:
Zonu Serum zonulin by ELISA with ELx800 reader / Kit IDK Zonulin ELISA ref. K 5601 / Immundiagnostik AG (Servibio)
TAS Total antioxidant status by enzymatic method with Hitachi 912 / Kit TOTAL ANTIOXIDANT STATUS (TAS) Ref NX2332 / Randox
ALAT SGPT by IFCC (International Federation of Clinical Chemistry) with pyridoxal phosphate at 37° C. on Cobas integra 400+ / Kit ALTL from Roche diagnostics
CHOG Total cholesterol by enzymatic colorimetry on Cobas Integra 400+/ Kit CHOL2 from Roche diagnostics
LDH Lactate dehydrogenase by UV test on Cobas integra 400+ / Kit LDHI2 from Roche diagnostics
Prealb Prealbumin by Immunoassay on Cobas integra 400§ / Kit PREA from Roche diagnostics
NMO Monocytes by flow cytometry (YUMIZEN H500 OT)
NPBA Polynuclear Basophils by flow cytometry (YUMIZEN H500 OT)

PN - Polynuclear Neutrophils (%) by flow cytometry (YUMIZEN H500 OT) The following parameters were measured in plasma samples from patients included in the HERACLES study at V1, V2, V3 and V4 with the associated methods:
Neopt Blood neopterin by ELISA with ELx800 reader / Kit Neopterin Elisa Ref RE9321 / IBL international GmbH
Citru Citrullin by Liquid Chromatography coupled to tandem Mass Spectrometry
Indox 3-Indoxylsulfate by Liquid Chromatography coupled to tandem Mass Spectrometry
ST2 : Suppression of Tumorigenicity 2 by ELISA with ELx800 reader / Kit human ST2/IL-33R immunoassay Quantikine ELISA ref DST200 / R&D Systems

The following parameters were measured in fecal samples from patients included in the HERACLES study at V1, V2, V3 and V4 with the associated methods:
Short chain fatty acids by Gas Chromatography-mass spectrometry
   ACETA_s AcetateBUTYR_s Butyrate
   ISOBUT_s Isobutyrate
   PROP_s Propionate
   VALERA_s Valerate
Biliary acids by Liquid Chromatography coupled to tandem Mass Spectrometry
   CA Cholic acid
   CDCA Chenodesoxycholic Acid
   DCA Desoxycholic Acid
   LCA Lithocholic Acid
CALturbo - Calprotectin by Immunoturbidimetry with Cobas Integra 400§ / Kit fCal Turbo Calprotecin Ref B KCAL-REST/ BUHLMANN
Zonu_s - stools zonulin by ELISA assay with ELx800 reader IDK Zonulin ELISA ref K5600 / Immunodiagnostik AG
Neopt_s - stools neopterin by ELISA assay with ELx800 reader / Kit Neopterin ELISA Ref RE59321 / IBL International GmbH

Using the completed list of host parameters, we employed a machine learning approach to extract from these features, the ones who weight the most in patient R vs NR stratification at each time point. R vs NR stratification was done as indicated above (HERACLES study).

### Machine Learning Modeling

The algorithm used during the training was Ridge logistic regression with internal cross-validation used to determine the strength of regularization. We chose logistic regression as a model that can be easily interpreted: it returns weights of each feature, positive, when a feature correlates with the response status, negative when it anti-correlates.

### Results

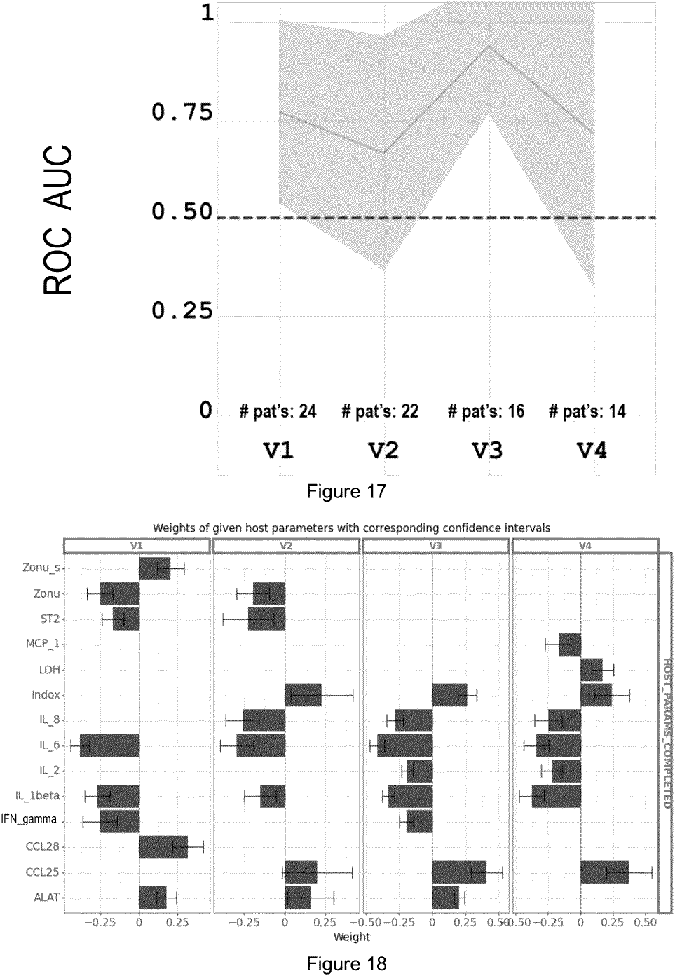

The overall results are expressed in terms of AUC (area under the ROC curve) which can be observed in FIG. 17.

This figure indicates the average AUC (grey line) surrounded by confidence intervals (light grey ribbon) for each time point. The number of patients included at each visit is also mentioned. The overall predictions, according to this figure are satisfying, especially at V1 (baseline) and V3 (after second product administration) where the min confidence interval does not cross the 0.5 AUC (dotted line) which can be interpreted as significant.

FIG. 18 shows the measured parameters that drive the most the prediction results.

For each point, the bar is oriented to the left when the values of the corresponding parameter are greater in NR patients, and to the right when the values are greater for R patients.

hese results allow to add several parameters to the ones previously noted as impactful for those patients. It includes at V1:
More in NR patients: IL-6, IL-1 beta, IFN_gamma, blood zonulin
More in R patients: CCL28

At V2, we have:
More in NR patients: IL-6, IL-8, IL-1 beta, blood zonulin
More in R patients: CCL25

At V3:
More in NR patients: IL-6, IL-8, IL-2, IL-1beta, IFN_gamma
More in R patients: CCL25

At V4:
More in NR patients: IL-6, IL-8, IL-2, IL-1 beta, MCP_1
More in R patients: CCL25

Thus, in addition to the markers for predicting FMT performance before administering the FMT product, these results show that the following markers can also be used after the FMT to assess whether said FMT was successful:
An increase in indoxyl sulfate concentration
IL8 below a certain threshold
CCL25 above a certain threshold, and/or
MCP_1 below a certain threshold.

### REFERENCES

Alexander JL et al. Gut microbiota modulation of chemotherapy efficacy and toxicity. Nature Reviews - Gastroenterology and hepatology. 2017.

Bacchetti De Gregoris T et al. Improvement of phylum- and class-specific primers for real-time PCR quantification of bacterial taxa. J. Microbiol. Methods, vol. 86, no 3, p. 351-356, September 2011.

Bolger AM et al. Trimmomatic: A Flexible Trimmer for Illumina Sequence Data. Bioinformatics. 2014.

Clarke, J. et al. Continuous base identification for single-molecule nanopore DNA sequencing. Nat. Nanotechnol. 2009: 4, 265-270.

Eid, J et al. Real-time DNA sequencing from single polymerase molecules. Science 2009: 323, 133-138.

Golob JL et al. Stool Microbiota at Neutrophil Recovery Is Predictive for Severe Acute Graft vs Host Disease After Hematopoietic Cell Transplantation. Clinical Infectious Diseases. 2017.

Guo X et al. Development of a real-time PCR method for Firmicutes and Bacteroidetes in faeces and its application to quantify intestinal population of obese and lean pigs. Lett. Appl. Microbiol., vol. 47, no 5, p. 367-373, 2008.

Holler E, et al. Metagenomic analysis of the stool microbiome in patients receiving allogeneic stem cell transplantation: loss of diversity is associated with use of systemic antibiotics and more pronounced in gastrointestinal graft-versus-host disease. Biol Blood Marrow Transplant 2014.

lida N et al. Commensal Bacteria Control Cancer Response to Therapy by Modulating the Tumor Microenvironment. Science 2013.

Jenq RR et al. Intestinal Blautia Is Associated with Reduced Death from Graft-versus-Host Disease. Biology of Blood and Marrow Transplantation. 2015.

Kakihana K, et al. Fecal microbiota transplantation for patients with steroid-resistant/dependent acute graft-versus-host disease of the gut. Blood 2016.

Langmead B et al. Fast Gapped-Read Alignment with Bowtie 2. Nat Methods. 2012.

Ma et al. Gut Microbiota Shapes the Efficiency of Cancer Therapy. Front Microbiol. 2019.

Magoč T et al. FLASH: Fast Length Adjustment of Short Reads to Improve Genome Assemblies. Bioinformatics. 2011.

Major-Monfried H et al. MAGIC biomarkers predict long-term outcomes for steroid-resistant acute GVHD. Blood. 2018 Jun 21;131(25):2846-2855.

Malard Florent, Gasc Cyrielle, Plantamura Emilie, Doré Joël. High gastrointestinal microbial diversity and clinical outcome in graft-versus-host disease patients. Bone Marrow Transplantation 2018.

Malíčková et al. Fecal zonulin is elevated in Crohn’s disease and in cigarette smokers. Pract Lab Med. 2017 Sep 23;9:39-44.

Peled JU et al. Microbiota as Predictor of Mortality in Allogeneic Hematopoietic-Cell Transplantation. New England Journal of Medicine 2020.

Qi X. et al. Treating Steroid Refractory Intestinal Acute Graft-vs.-Host Disease With Fecal Microbiota Transplantation: A Pilot Study. Front Immunol. 2018.

Rhoads A and Au KF. PacBio Sequencing and Its Applications. Genomics Proteomics Bioinformatics. 2015 Oct;13(5):278-89.

Rognes T et al. VSEARCH: A Versatile Open Source Tool for Metagenomics. PeerJ. 2016.

Shouval R et al. Repeated Courses of Orally Administered Fecal Microbiota Transplantation for the Treatment of Steroid Resistant and Steroid Dependent Intestinal Acute Graft Vs. Host Disease: A Pilot Study (NCT 03214289). Blood 2018.

Spindelboeck W, et al. Repeated fecal microbiota transplantations attenuate diarrhea and lead to sustained changes in the fecal microbiota in acute, refractory gastrointestinal GVHD. Haematologica.2017.

Taur Y et al. Intestinal Domination and the Risk of Bacteremia in Patients Undergoing Allogeneic Hematopoietic Stem Cell Transplantation”. Clin Infect Dis. 2012.

Taur Y et al. The effects of intestinal tract bacterial diversity on mortality following allogeneic hematopoietic stem cell transplantation”. Blood 2014.

van Lier et al. Fecal Microbiota Transplantation Can Cure Steroid-Refractory Intestinal Graft-Versus-Host Disease. Biology of Blood and Marrow Transplantation. 2019

Vokurka et al. Serum citrulline levels as a marker of enterocyte function in patients after allogeneic hematopoietic stem cells transplantation - a pilot study. Med Sci Monit 2013; 19:81-85.

Weber D et al. Low urinary indoxyl sulfate levels early after transplantation reflect a disrupted microbiome and are associated with poor outcome. Blood. 2015.

Yang Y-W et al. Use of 16S rRNA Gene-Targeted Group-Specific Primers for Real-Time PCR Analysis of Predominant Bacteria in Mouse Feces, Appl. Environ. Microbiol., vol. 81, no 19, p. 6749-6756, October 2015.

1. A method for assessing whether a subject in need of a complementation of his/her gastrointestinal microbiota with live microorganisms can benefit from said complementation, comprising the steps of:
a1. measuring, in a gastrointestinal biological sample from said subject, the abundances of bacteria associated with a good prognosis, wherein said bacteria belong to at least one category selected from the group consisting of:
Firmicutes phylum;
Bacilli and Actinobacteria classes;
Bacillales, Lactobacillales and Micrococcales orders;
Staphylococcaceae,, Lactobacillaceae, and Micrococcaceae families; and
Staphylococcus, Lactobacillus, Melissococcus and Arthrobacter genera; and
a2. determining a first value (#G), corresponding to a weighted sum of the abundances measured in step a1; and/or
b1. measuring, in a gastrointestinal biological sample from said subject, the abundances of bacteria associated with a bad prognosis, wherein said bacteria belong to at least one category selected from the group consisting of:
Bacteroidetes and Proteobacteria phyla;
Bacteroidia class;
Bacteroidales and Enterobacteriales orders;
Bacteroidaceae, Porphyromonadaceae, Acidaminococcaceae, Lachnospiraceae, Ruminococcaceae, Clostridiaceae, Prevotellaceae and Erysipelotrichaceae families; and
Bacteroides, Escherichia, Shigella, Ruminococcus, Faecalibacterium, Dorea, Coprococcus, Blautia, Alistipes, Subdoligranulum, Roseburia, Parabacteroides and Lachnospira genera; and
b2. determining a second value (#B), corresponding to a weighted sum of the abundances measured in step b1; and
c. using the results obtained in step a and/or in step b, calculating at least one score (#R) selected from the group consisting of #R1=#G, #R2=1:#B and/or #R3=#G:#B; and
d. comparing each score obtained in step c to one or several reference values, wherein if #R1, #R2 and/or #R3 is(are) superior to the reference value(s), the subject is likely to benefit from the complementation with live microorganisms, and if #R1, #R2 and/or #R3 is(are) inferior to the reference value(s), the subject needs a treatment prior to the complementation with live microorganisms for the microorganisms to successfully engraft in the subject’s gut.

2. The method of claim 1, wherein said complementation with live microorganisms is a fecal microbiota transplant (FMT).

3. The method according to claim 1, wherein the subject suffers from a graft versus host disease (GvHD) following allogeneic hematopoietic stem cell transplantation (allo-HSCT).

4. The method according to claim 3, wherein the subject suffers from an acute, steroid-refractory graft versus host disease (SR-aGvHD) following allogeneic hematopoietic stem cell transplantation (allo-HSCT).

5. The method according to claim 3 wherein the subject suffers from a gastrointestinal GvHD (GI GvHD).

6. The method according to claim 1, wherein
(i) #G= Firmicutes, #B= Bacteroidetes and #R=#G:#B; and/or
(ii) #G= Firmicutes phylum excluding Acidaminococcaceae and Lachnospiraceae families, #B= Bacteroidetes and #R=#G:#B; and/or
(iii) #G= Firmicutes + Actinobacteria, #B= Bacteroidetes and #R=#G:#B; and/or
(iv) #G= Actinobacteria + Firmicutes excluding Acidaminococcaceae and Lachnospiraceae families, #B= Bacteroidetes and #R=#G:#B; and/or
(v) #G= Firmicutes, #B= Bacteroidetes + Proteobacteria and #R=#G:#B; and/or
(vi) #G= Firmicutes phylum excluding Acidaminococcaceae and Lachnospiraceae families, #B= Bacteroidetes + Proteobacteria and #R=#G:#B; and/or
(vii) #G= Firmicutes + Actinobacteria, #B= Bacteroidetes + Proteobacteria and #R=#G:#B; and/or
(viii) #G= Actinobacteria + Firmicutes excluding Acidaminococcaceae and Lachnospiraceae families, #B= Bacteroidetes + Proteobacteria and #R=#G:#B; and/or
(ix) #G= Firmicutes and #R=#G; and/or
(x) #G= Firmicutes phylum excluding Acidaminococcaceae and Lachnospiraceae families, and #R=#G; and/or
(xi) #G= Firmicutes + Actinobacteria and #R=#G; and/or
(xii) #G= Actinobacteria + Firmicutes excluding Acidaminococcaceae and Lachnospiraceae families and #R=#G; and/or
(xiii) #B= Bacteroidetes and #R=1:#B; and/or
(xiv) #B= Bacteroidetes + Proteobacteria and #R=1:#B; and/or
(xv) #G= Bacilli + optionally Actinobacteria, #B= Bacteroidia + optionally Gammaproteobacteria + optionally Negavicutes + optionally Clostridia and #R=#G:#B; and/or
(xvi) #G= Bacillales + Lactobacillales + Micrococcales, #B= Bacteroidales + Enterobacteriales + optionally Selenomonadales + optionally Clostridiales and #R=#G:#B; and/or
(xvii) #G= Staphylococcaceae, + Lactobacillaceae + Micrococcaceae + optionally Enterococcaceae, #B= Bacteroidaceae + Porphyromonadaceae + Acidaminococcaceae + Lachnospiraceae + optionally Enterobacteriaceae and #R=#G:#B; and/or
(xviii) #G= Staphylococcus + Lactobacillus + Melissococcus + Arthrobacter, #B= Bacteroides + Escherichia + Shigella and #R=#G:#B; and/or
(xix) #G= Bacilli + Micrococcales, #B= Bacteroidia + Enterobacteriales + Acidaminococcaceae + Lachnospiraceae and #R=#G:#B; and/or
(xx) #B= Lachnospiraceae + Ruminococcaceae + Clostridiaceae, Prevotellaceae + Erysipelotrichaceae and #R=1:#B; and/or
(xxi) #B= Bacteroides + Ruminococcus + Faecalibacterium + Dorea + Coprococcus + Blautia + Alistipes + Subdoligranulum + Roseburia + Parabacteroides + Lachnospira and #R=1:#B.

7. The method according to claim 1, wherein said gastrointestinal biological sample is a rectal swab or a feces sample.

8. The method according to claim 1, wherein bacteria are quantified by qPCR, 16S sequencing, whole metagenomics sequencing or by microarray.

9. The method according to claim 1, wherein #B= Lachnospiraceae + Ruminococcaceae + Clostridiaceae + Prevotellaceae + Erysipelotrichaceae and #R=1:#B>100 indicates that the subject is likely to benefit from the complementation with live microorganisms.

10. The method according to claim 1, wherein #B= Bacteroides + Ruminococcus + Faecalibacterium + Dorea + Coprococcus + Blautia, Alistipes + Subdoligranulum + Roseburia + Parabacteroides + Lachnospira and #R=1:#B>50 indicates that the subject is likely to benefit from the complementation with live microorganisms.

11. The method according to claim 1, further comprising:
i. from at least one biological sample from the subject, measuring one or several prognostic markers selected from the concentrations of cholesterol, indoxylsulfate, fecal zonulin, citrullin, prealbumin, suppressor of tumorigenicity-2 (ST2), regenerating-islet-derived protein 3-α (REG3α), IL-6, IL-1β, IFNγ, CCL28 and/or IL-2;
ii. comparing the values obtained in step a to reference values, wherein:
fecal zonulin concentration superior to a reference value;
citrullin concentration superior to a reference value;
prealbumin concentration superior to a reference value;
cholesterol concentration superior to a reference value;
indoxylsulfate concentration inferior to a reference value;
ST2 concentration inferior to a reference value;
REG3α concentration inferior to a reference value;
IL-6 concentration inferior to a reference value;
IL-2 concentration inferior to a reference value;
IL-1β concentration inferior to a reference value;
IFNγ concentration inferior to a reference value; and/or
CCL28 concentration superior to a reference value;
are additional indicators of good prognosis.

12. The method of claim 11, wherein:
fecal zonulin concentration is measured in a rectal swab or a feces sample; and
the other prognostic markers are measured in blood, plasma or serum.

13. The method of claim 1, further comprising administering an FMT product to a subject for whom the test by the method of claim 1 indicated that the subject is likely to benefit from a complementation with live microorganisms.

14. A kit for performing the method according to claim 1, comprising primers specific for the bacterial taxa for which the abundance is measured.

15. A method for treatment of GvHD comprising administering an FMT product to a subject for whom the test by the method of claim 1 indicated that the subject is likely to benefit from a complementation with live microorganisms.
